(19) United States
(12) Patent Application Publication
(10) Pub. No.: US 2021/0395242 A1
(43) Pub. Date: Dec. 23, 2021

(54) HETEROCYCLIC COMPOUNDS AS AHR MODULATORS

(71) Applicant: JAGUAHR THERAPEUTICS PTE LTD, Singapore (SG)

(72) Inventors: Antonio METE, Leicestershire (GB); James HITCHIN, Nottingham (GB); Mark GRAHAM, Leicestershire (GB)

(21) Appl. No.: 17/272,299

(22) PCT Filed: Aug. 30, 2019
(86) PCT No.: PCT/EP2019/073218

§ 371 Date: Feb. 27, 2021

(30) Foreign Application Priority Data
Aug. 31, 2018 (SG) 10201807515T

(51) Int. Cl. C07D 417/12; C07D 403/12; C07D 413/12

(52) U.S. Cl. CPC C07D 417/12; C07D 413/12; C07D 403/12

(57) ABSTRACT

The present invention relates compounds of the general formula (I) or (III) which are ARH inhibitors, methods for preparing said compounds, pharmaceutical compositions and combinations comprising said compounds and the use of said compounds and pharmaceutical compositions for the treatment or prevention of diseases, in particular cancer or conditions with dysregulated immune functions, or other conditions associated with aberrant AHR signalling, as a sole agent of in combination with other active ingredients. Such compounds may also be of utility in the expansion of hematopoietic stem cells (HSCs) and the use of HSCs in autologous or allogenic transplantation for the treatment of patients with inherited immunological and autoimmune diseases and diverse hematopoietic disorders.

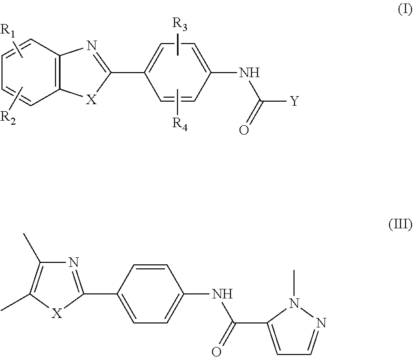

The present invention covers compounds of the general formula (I) as described and defined herein, methods for preparing said compounds, pharmaceutical compositions and combinations comprising said compounds and the use of said compounds or pharmaceutical compositions for the treatment or prevention of diseases, in particular cancer or conditions with dysregulated immune functions, or other conditions associated with aberrant AHR signalling. The compounds disclosed herein may be employed as a sole agent of in combination with other active ingredients. Such compounds may also have utility in the expansion of hematopoietic stem cells (HSCs) and the use of HSCs in autologous or allogenic transplantation for the treatment of patients with inherited immunological and autoimmune diseases and diverse hematopoietic disorders.

### BACKGROUND

The aryl hydrocarbon receptor (AhR) is a ligand-activated factor that belongs to the family of the basic helix-loop-helix-Per/ARNT/Sim family. Following ligand binding in the cytoplasm, AhR dissociates from its complex with Hsp90 and the AhR-interacting protein, XAP2, allowing ligated AhR to translocate to the nucleus. There, AhR dimerizes with the AhR nuclear translocator (ARNT), that then binds to xenobiotic response elements (XREs) promoting the up- or down-regulation of a multitude of target genes in many different tissues. The AhR is best known for binding to environmental toxins and inducing various members of the cytochrome P450 family including CYP1A1, CYP1A2 and CYP1B1 required for their elimination. Activation of AhR by xenobiotics has demonstrated that this receptor plays a role in a range of physiological processes including embryogenesis, tumourigenesis and inflammation (Esser & Rannug, Pharmacol Rev, 2015, 67:259; Roman et al., Pharmacol Ther, 2018, 185:50).

AhR is expressed in many immune cell types including dendritic cells (DCs), macrophages, T cells, NK cells and B cells and plays an important role in immunoregulation (Quintana & Sherr, Pharmacol Rev, 2013, 65:1148; Nguyen et al., Front Immunol, 2014, 5:551). The toxic/adverse effects of classical exogenous AhR agonists, such as 2,3,7,8-Tetrachlorodibenzo-p-dioxin (TCDD) are well known and include profound immunosuppression and initiation of malignancy (Esser et al., Trends Immunol, 2009, 30:447; Feng et al., Biochimica et Biophysica Acta, 2013, 1836:197). Physiological effects of AhR agonists on immune cells include promotion of regulatory T cell (Treg) generation (Pot, Swiss Med Wkly, 2012, 142:w13592) and modulation of Th17 cell differentiation and activation (Baricza et al., Cell Mol Life Sci, 2016, 73:95). AhR also modulates the function of antigen presenting cells such as dendritic cells and macrophages. AhR activation decreases the expression of class II major histocompatibility complex and co-stimulatory molecules and also the production of Th1 and Th17 polarizing cytokines by dendritic cells (Mezrich et al., J Immunol, 2010, 185:3190; Nguyen et al., Proc Natl Acad Sci USA, 2010, 107:19961; Quintana et al., 2010 Proc Natl Acad Sci USA, 107:20768). Indeed, AhR activation boosts the ability of DCs to promote the differentiation of Tregs (Jurado-Manzano et al., 2017, Immunol Lett, 190:84).

In addition to xenobiotics, the AhR can also bind metabolic products of tryptophan degradation including kynurenine (KYN) and kynurenic acid (KYNA). Indoleamine 2,3 dioxygenase 1 and 2 (IDO1/IDO2) and tryptophan 2,3-dioxygenase 2 (TDO2) catalyse the commitment step of the KYN metabolic pathway and are expressed in immune cells (IDO1) and a range of cancer cells (IDO1 and TDO2)(Pilotte et al., Proc Nat Acad Sci, 2012, 109:2497). Inhibitors of IDO1 have attracted much interest as potential new treatments to stimulate the immune system to recognize and eliminate cancer cells (Cheong & Sun, Trends Pharmacol Sci, 2018, 39:307). Traditionally the immunosuppressive effect of IDO1 has been attributed mainly to reduced levels of tryptophan, which activates the kinase GCN2 (general control non-derepressible 2) and inhibits T cell proliferation/activation both in tumour draining lymph nodes, lymph nodes and in the tumour micro-environment. More recently it has become apparent that some of the efficacy of IDO inhibitors may be the result of decreased production of AhR agonists. These endogenously generated AhR agonists have been shown to elicit a range of effects on immune cells including upregulation of IDO1 in dendritic cells (Julliard et al., Front Immunol, 2014, 5:458), inhibition of human T cell proliferation (Frumento et al., J Exp Med, 2002; 196:459; Terness et al., J Exp Med, 2002; 196: 447; Opitz et al., Nature, 2011, 478:197) and up-regulation of PD-1 expression in cytotoxic T lymphocytes (Liu et al., Cancer Cell, 2018; 33:480). As highlighted above, IDO1 is not the only source of endogenous AhR agonists. TDO2 is predominately expressed in the liver but it is also constitutively expressed in some cancers, notably malignant glioma, hepatocellular carcinoma, melanoma, bladder, breast, lung and colorectal cancer (Opitz et al., Nature, 2011, 478:197; Pilotte et al., Proc Nat Acad Sci, 2012, 109:2497; D'Amato et al., Cancer Res, 2015, 75(21):4651; Hsu et al., Oncotarget, 2016, 7(19): 27584; Chen et al., Dis Markers, 2016, 2016:8169724). Such data suggests that AhR antagonists may have broader efficacy than selective IDO-1 inhibitors as they will attenuate endogenous AhR agonist signalling regardless of its source.

In addition, to their effects on immune cells such endogenous agonists have also been implicated in cancer progression via direct effects on the tumour. For example, KYN increases human glioblastoma cell survival and migration (Opitz et al., Nature, 2011, 478:197). Several other studies also implicate the AhR in cancer progression in the absence of environmental ligands. The AhR-repressor (AHRR) protein acts as a tumour suppressor gene in several human cancers (Zudaire et al., J Clin Invest, 2008, 118:640). AhR expression and “constitutive” (endogenous ligand-driven) activity in breast cancer cells correlate with tumour aggressiveness (Schlezinger et al., Biol Chem, 2006, 387:1175; Yang et al., J Cell Biochem, 2008, 104:402) and control expression of genes associated with tumour invasion (Yang et al., Oncogene, 2005, 24:7869). Ectopic AhR expression in non-malignant human mammary epithelial cells induces an epithelial-to-mesenchymal transition and a >50% increase in cell growth rates (Brooks & Eltom, Curr Cancer Drug Targets, 2011, 11:654) and AhR knockdown induced gene changes in human breast cancer cell lines consistent with a mesenchymal to epithelial cell reversion to a less aggressive phenotype (Narasimhan et al., Int J Mol Sci, 2018, 19:1388). AhR antagonists or AhR knockdown has been shown to reduce proliferation, survival, invasiveness and migration of human breast cancer cells in culture (Parks et al., Mol Pharmacol, 2014, 86:593; D'Amato et al., Cancer Res, 2015, 75(21):4651; Narasimhan et al., Int J Mol Sci, 2018, 19:1388) and to reduce survival of glioblastoma cells (Gramatzki et al., Oncogene, 2009, 28:2593; Opitz et al., Nature, 2011, 478:197; Guastella et al., J Neuro-oncol, 2018, in press). Finally, AhR antagonists block the formation of tumourspheres (Stanford et al., Mol Cancer Res, 2016, 14:696) which are formed by cancer stem cells (CSCs), a subset of tumour cells that drive the initiation, progression and metastasis of tumours.

Thus, AhR agonists released from immune cells and from tumour cells act in an autocrine and paracrine fashion to promote tumour growth. Agents that reduce or block these effects may therefore find utility in the treatment of cancer and/or conditions with dysregulated immune functions.

WO2017/202816 relates to compounds and compositions for the treatment or prophylaxis of cancer or conditions with dysregulated immune responses or other disorders associated with aberrant AhR signalling. In particular, WO2017/202816 relates inter alia to heterocyclic compounds capable of inhibiting AhR function.

WO2010/059401 relates to compounds and compositions for expanding the number of CD34+ cells for transplantation. In particular, WO2010/059401 relates inter alia to heterocyclic compounds capable of downregulating the activity and/or expression of AhR.

WO2012/015914 relates to compositions and methods for modulating AhR activity. In particular, WO2012/015914 relates inter alia to heterocyclic compounds that modulate AhR activity for use in therapeutic compositions to inhibit cancer cell proliferation and tumour cell invasion and metastasis.

### SUMMARY OF THE DISCLOSURE

The present disclosure provides benzazole compounds of general formula (I) which inhibit the AhR. The disclosure is summarised in the following paragraphs:
1. A compound of formula (I):

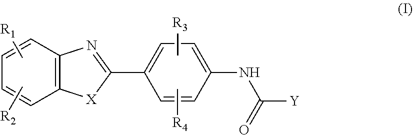

wherein
X is a heteroatom selected from O, S or NR⁷;
R¹ is H, C₁₋₃ alkyl, —C(O)OC₁₋₃alkyl, —C(O)NR⁵R⁶C₁₋₃ alkyl, Halogen (such as F, Cl, Br or I, in particular Cl), C₁₋₃hydroxyalkyl (such as (—CH₂)ₙOH), —CN, C₁₋₃haloalkyl or C₁₋₃alkoxy;
R² is H, C₁₋₃ alkyl, —C(O)OC₁₋₃alkyl, —C(O)NR⁵R⁶C₁₋₃ alkyl, Halogen (such as F, Cl, Br or I, in particular Cl), (—CH₂)ₙOH, or —CN;
R³ is H or C₁₋₃ alkyl, Halogen;
R⁴ is H or C₁₋₃ alkyl, Halogen;
R⁵ is selected from H or C₁₋₃alkyl;
R⁶ is selected from H or C₁₋₃ alkyl;
R⁷ is selected from H or C₁₋₃ alkyl;
R⁸ is selected from no substituent, H, C₁₋₃ alkyl and (CH₂)mOC₁₋₃ alkyl, (—CH₂)ₙOH;
n is 2 or 3;
m is 2 or 3;
q is 1 or 2;
Y is a 5 or 6 membered heteroaryl comprising at least one heteroatom —NR⁸ and at least one further heteroatom (for example 1 or 2 further heteroatoms) independently selected from S, O or N said heteroaryl is optionally substituted by one, two or three groups independently selected from halogen, hydroxyl, C₁₋₆ alkyl, C₁₋₆ alkoxy, C₁₋₆ haloalkyl, amino, C₁₋₄ mono and di-alkyl amino, C₁₋₄ mono or di-acyl amino, S(O)_{q}C₁₋₆ alkyl, C₀₋₆ alkylC(O)C₁₋₆ alkyl or C₀₋₆ alkylC(O)C₁₋₆ heteroalkyl; or a pharmaceutically acceptable salt thereof.
2. A compound of formula (I) or a pharmaceutically acceptable salt thereof according to paragraph 1, wherein X is S.
3. A compound of formula (I) or a pharmaceutically acceptable salt thereof according to paragraph 1, wherein X is O.
4. A compound of formula (I) or a pharmaceutically acceptable salt thereof according to paragraph 1, wherein X is —NR⁷ as shown in formula (II):

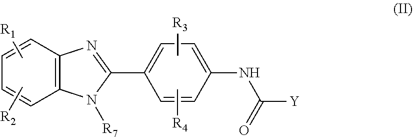

5. A compound of formula (I) or (II) or a pharmaceutically acceptable salt thereof according to paragraph 4, wherein the R⁷ is H, —CH₃ or —CH₂CH₃, for example H or —CH₂CH₃ such as H.
6. A compound of formula (I) or (II) or a pharmaceutically acceptable salt thereof according to any one of paragraphs 1 to 5, wherein R² is hydrogen.
7. A compound of formula (I) or (II) or a pharmaceutically acceptable salt thereof according to any one of paragraphs 1 to 6, wherein R⁴ is hydrogen.
8. A compound of formula (I) or (II) or a pharmaceutically acceptable salt thereof according to any one of paragraphs 1 to 7, wherein R¹ is H, C₁₋₃ alkyl, —C(O)OC₁₋₃alkyl, —C(O)NR⁵R⁶C₁₋₃ alkyl, Halogen (such as F, Cl, Br or I, in particular Cl), (—CH₂)ₙOH, —CN, C₁₋₃haloalkyl or C₁₋₃ alkoxy.
9. A compound of formula (I) or (II) according to any one of paragraphs 1 to 7, wherein R¹ is selected from H, —CH₃, CO₂CH₃, CN, —C(O)NH₂, —C(O)NHCH₃, —C(O)NHCH₂CH₃, —C(O)NCH₃CH₃, Cl, —CH(CH₃)₂OH and —CH₂OH.
10. A compound of formula (I) or (II) according to any one of paragraphs 1 to 9 or a pharmaceutically acceptable salt thereof, wherein R¹ is selected from H, —CH₃, CO₂CH₃, —C(O)NH₂, —C(O)NHCH₃, —C(O)NCH₃CH₃, Cl, and —CH₂OH.
11. A compound of formula (I) or (II) according to paragraph 10 or a pharmaceutically acceptable salt thereof wherein R¹ is selected from H, —CH₃, —C(O)₂CH₃, —C(O)NH₂, and —CHOH.
12. A compound of formula (I), (III) or (III) according to any one of paragraphs 7 to 11 or a pharmaceutically acceptable salt thereof, wherein R¹ is selected from —CH₃, —C(O)₂CH₃, —C(O)NH₂, and CHOH.
13. A compound of formula (I) or (II) according to any one of paragraphs 1 to 12 or a pharmaceutically acceptable salt thereof, wherein R² is selected from H or CH₃.
14. A compound of formula (I) or (II) according to any one of paragraphs 1 to 13, or a pharmaceutically acceptable salt thereof, wherein Y is a five membered heteroaryl.
15. A compound of formula (I) or (II) according to any one of paragraphs 1 to 14, or a pharmaceutically acceptable salt thereof, wherein Y is pyrazolyl, such as pyrazol-5-yl.
16. A compound of formula (I) or (II) according to any one of paragraphs 1 to 15, or a pharmaceutically acceptable salt thereof, wherein Y is 1-methyl-1H-pyrazol-5-yl or 1-(2-methoxyethyl)-1H-pyrazol-5-yl.
17. A compound according to any one of paragraphs 1 to 16, wherein R⁸ is selected from H, —CH₃, —CH₂CH₂OCH₃.
18. A compound according to paragraph 17, or a pharmaceutically acceptable salt thereof, wherein R⁸ is H.
19. A pharmaceutical composition comprising a compound of formula (I) or (II) or a pharmaceutically acceptable salt thereof according to any one of paragraph 1 or 19 and an excipient, diluent or carrier.
20. A compound of formula (I) or (II) or a pharmaceutically acceptable salt thereof according to any one of claims 1 to 18 or a pharmaceutical composition according to claim 19, for use in treatment, for example for use in the treatment of cancer.
21. A compound of formula (I) or (II) or a pharmaceutically acceptable salt thereof according to any one of claims 1 to 18 or a pharmaceutical composition according to claim 19, for use in the manufacture of a medicament for the treatment of cancer.
22. A method of treating a patient comprising administering a therapeutically effective amount of a compound of formula (I) or (II) or a pharmaceutically acceptable salt thereof according to any one of claims 1 to 18 or a pharmaceutical composition according to claim 19.
23. A compound of formula (III):

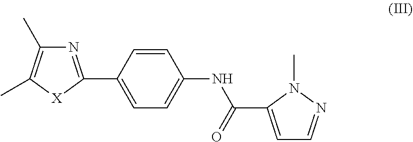

wherein X is defined for compounds of formula (I) or a pharmaceutically acceptable salt thereof.
24. A compound of formula (III) wherein X is O.
25. A pharmaceutical composition comprising a compound of formula (III) according to paragraph 23 or 24 and an excipient, diluent or carrier.
26. A compound of formula (III) according to paragraph 23 or 24 or a composition according to paragraph 25 for use in treatment.
27. A compound of formula (III) according to paragraph 23 or 24 or a composition according to paragraph 25 for use in the treatment of cancer.
28. A compound of formula (III) or a pharmaceutically acceptable salt thereof according to paragraph 23 or 24 or a composition according to paragraph 25, for use in the manufacture of a medicament for the treatment of cancer.
29. A method of treating a patient comprising administering a therapeutically effective amount of a compound of formula (III) or a pharmaceutically acceptable salt according to paragraph 23 or 24 or a pharmaceutical composition according to paragraph 25.

In particular, the compounds of the present invention have surprisingly been found to effectively inhibit AhR. The data supports that the compounds may be useful for the treatment or prophylaxis of conditions where exogenous and endogenous AhR ligands induce dysregulated immune responses, for example: uncontrolled cell growth; proliferation and/or survival of tumour cells; immunosuppression for example in the context of cancer; inappropriate cellular immune responses; and inappropriate cellular inflammatory responses.

In one embodiment the compounds of the present disclosure are useful in the treatment of cancer for example, liquid and/or solid tumours, and/or metastases thereof. Examples of cancers include head and neck cancer (such as brain tumours and brain metastases), cancer of the thorax including non-small cell and small cell lung cancer, gastrointestinal cancer (including stomach, oesophageal, colon, and colorectal), biliary tract cancer, pancreatic cancer, liver cancer, endocrine cancer, breast cancer, ovarian cancer, bladder cancer, kidney cancer and prostate cancer, skin cancer.

In one embodiment the cancer is an epithelial cancer. In one embodiment the cancer is a sarcoma. In one embodiment the cancer is a metastatic.

### DETAILED DISCLOSURE

C₁₋₃ alkyl (alkyl group) as employed herein refers to straight or branched chain alkyl, for example methyl, ethyl, propyl or isopropyl.

Halogen as employed herein includes fluoro, chloro, bromo or iodo.

Haloalkyl refers to an alkyl moiety wherein one to six hydrogens have been replaced by a halogen, for example 1, 2, 3, 4, 5 or 6, such as 1, in particular an alkyl bearing one chloro.

Alkoxy as used herein refers to a straight or branched chain alkoxy, for example methoxy, ethoxy, propoxy etc. Alkoxy as employed herein also extends to embodiments in which the oxygen atom is located within the alkyl chain, for example —C₁₋₃ alkylOC₁₋₃ alkyl, or —C₁ₒᵣ₂ alkylOC₁ₒᵣ₂ alkyl, such as —CH₂CH₂OCH₃ or —CH₂OCH₃. Thus, in one embodiment the alkoxy is linked through carbon to the remainder of the molecule. In one embodiment the alkoxy is linked through oxygen to the remainder of the molecule, for example —OC₁₋₃ alkyl. In one embodiment the disclosure relates to straight chain alkoxy.

Hydroxyalkyl as employed herein refers to an alkyl moiety where 1 or 2 (such as 1) hydrogen has been replaced by a hydroxyl group (—OH).

Heteroalkyl as employed herein refers to an alkyl group wherein one or more carbon atoms, such as 1 or 2 carbons are replaced by a heteroatom independently selected from S, N and O.

CO represents carbonyl.

The compounds of the present disclosure can be prepared by methods described herein.

5 or 6 membered heteroaryl as employed herein is a ring containing 5 or 6 atoms wherein at least one atom is a heteroatom, for example selected from nitrogen, oxygen or sulphur, such as pyrrole, pyrazole, imidazole, thiophene, oxazole, isothiazole, thiazole, pyridine, pyridazine, pyrimidine, pyrazine, triazine, thiopyran, oxazine and thiazine, such as pyrrole, pyrazole and pyridine and pyrimidine, in particular oxazole or pyrazole.

In one embodiment R¹ or R² (such as R¹) is located on the carbon beta to X.

In one embodiment R¹ or R² (such as R²) is located on the carbon beta to the N in the bicyclic ring system.

In one embodiment R³ or R⁴ is located alpha to bond linking the phenyl ring to the bicyclic system containing X.

Specific compounds of the present disclosure include:
N-(4-(1-ethyl-1H-benzo[d]imidazol-2-yl)phenyl)-1-methyl-1H-pyrazole-5-carboxamide;
N-(4-(benzo[d]oxazol-2-yl)phenyl)-1-(2-methoxyethyl)-1H-pyrazole-5-carboxamide;
Methyl 2-(4-(1-methyl-1H-pyrazole-5-carboxamido)phenyl)benzo[d]oxazole-7-carboxylate;
N-methyl-2-(4-(1-methyl-1H-pyrazole-5-carboxamido)phenyl)benzo[d]oxazole-7-carboxamide;
1-methyl-N-(4-(7-methylbenzo[d]oxazol-2-yl)phenyl)-1H-pyrazole-5-carboxamide;
N-(4-(benzo[d]oxazol-2-yl)-3-methylphenyl)isoxazole-3-carboxamide;
N-(3-methyl-4-(7-methylbenzo[d]oxazol-2-yl)phenyl) isoxazole-3-carboxamide;
1-methyl-N-(3-methyl-4-(7-methylbenzo[d]oxazol-2-yl)phenyl)-1H-pyrazole-5-carboxamide;
N-(4-(benzo[d]oxazol-2-yl)phenyl)-1-methyl-1H-pyrazole-5-carboxamide;
N-(4-(7-chlorobenzo[d]oxazol-2-yl)phenyl)-1-methyl-1H-pyrazole-5-carboxamide;
N-(2-(1H-indol-3-yl)ethyl)-2-(4-(1-methyl-1H-pyrazole-5-carboxamido)phenyl) benzo[d]oxazole-7-carboxamide;
N-(4-(7-(hydroxymethyl)benzo[d]oxazol-2-yl)phenyl)-1-methyl-1H-pyrazole-5-carboxamide;
2-(4-(1-methyl-1H-pyrazole-5-carboxamido)phenyl) benzo[d]oxazole-7-carboxamide;
N-Ethyl-2-(4-(1-methyl-1H-pyrazole-5-carboxamido) phenyl)benzo[d]oxazole-7-carboxamide;
N-(4-(7-Cyanobenzo[d]oxazol-2-yl)phenyl)-1-methyl-1H-pyrazole-5-carboxamide;
1-methyl-N-(4-(4-methylbenzo[d]oxazol-2-yl)phenyl)-1H-pyrazole-5-carboxamide;
N-(4-(7-(2-hydroxypropan-2-yl)benzo[d]oxazol-2-yl)phenyl)-1-methyl-1H-pyrazole-5-carboxamide;
N-(4-(7-chloro-1H-benzo[d]imidazol-2-yl)phenyl)-1-methyl-1H-pyrazole-5-carboxamide;
N-(4-(1H-benzo[d]imidazol-2-yl)phenyl)-1-methyl-1H-pyrazole-5-carboxamide;
N-(4-(benzo[d]thiazol-2-yl)phenyl)-1-methyl-1H-pyrazole-5-carboxamide;
N-(4-(benzo[d]oxazol-2-yl)-3-methylphenyl)-1-methyl-1H-pyrazole-5-carboxamide;
1-methyl-N-(3-methyl-4-(4-methylbenzo[d]oxazol-2-yl)phenyl)-1H-pyrazole-5-carboxamide; and
1-(2-methoxyethyl)-N-(4-(7-methylbenzo[d]oxazol-2-yl)phenyl)-1H-pyrazole-5-carboxamide.

Compounds of formula (I) can be made by the following generic route:

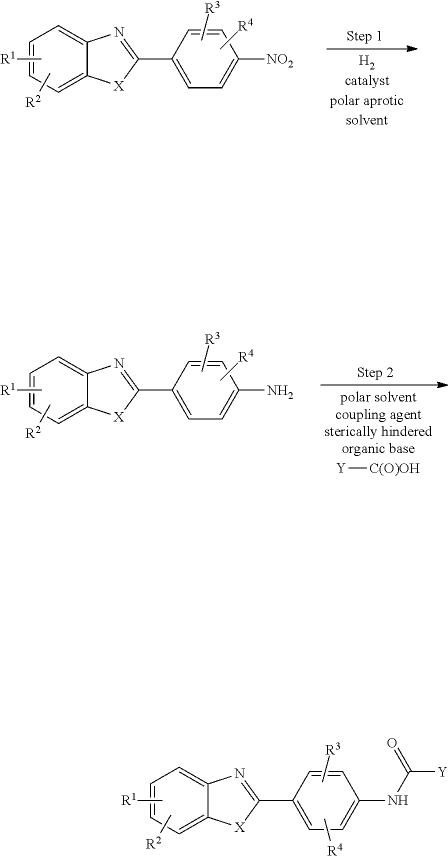

wherein:
X, Y, R¹, R², R³ and R⁴ are define above for compounds of formula (I).
In one embodiment the catalyst in step 1 is a metal such as palladium or platinum.
In one embodiment the polar aprotic solvent in step 1 is dimethyl formamide.
In one embodiment the polar solvent in step 2 is an alcohol, such as ethanol.
In one embodiment the coupling agent is HATU (1-[Bis(dimethylamino)methylene]-1H-1,2,3-triazolo[4,5-b]pyridinium 3-oxid hexafluorophosphate), which is generally employed in combination with a catalytic amount of DMAP (4-dimethylaminopyridine).
In one embodiment the sterically hindered organic base in step 2 is triethylamine.
The intermediate nitro compound wherein X is S or O can be prepared by one of the two generic routes given below:

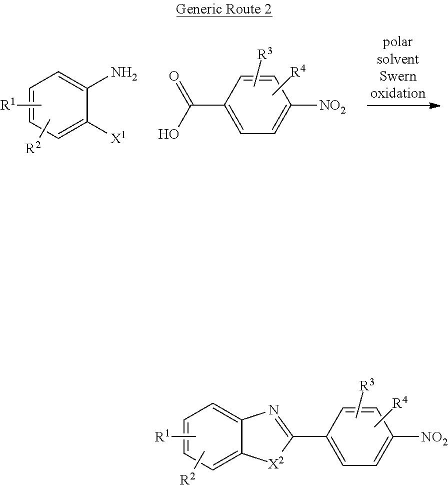

wherein
R¹, R², R³ and R⁴ are defined above for compounds of formula (I), X¹ is SH or OH and X² is S or O.
In one embodiment the polar solvent is DCM (dichloromethane) and a small amount of DMF (dimethyl formamide).
The Swern oxidation generally employs oxalyl chloride and an organic base, such as triethylamine.

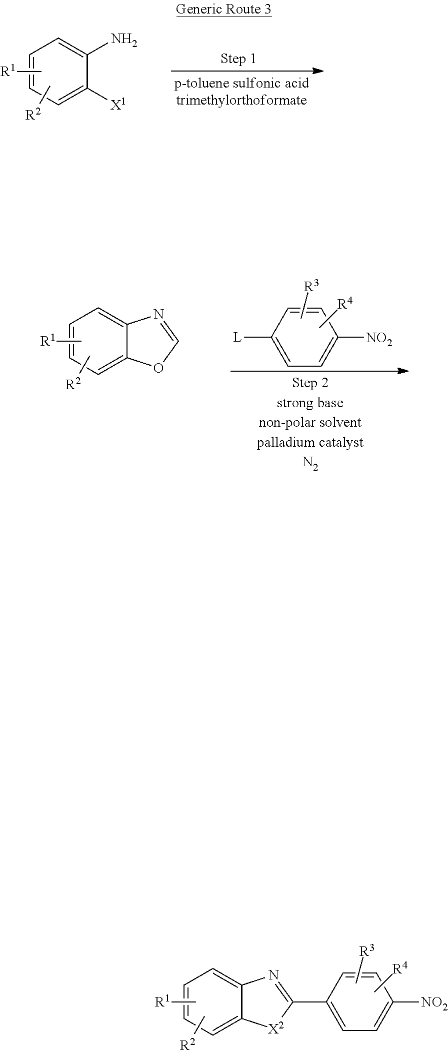

wherein
R¹, R², R³ and R⁴ are defined above for compounds of formula (I), X¹ is SH or OH, X² is S or O, and L is a leaving group.
The leaving group L is generally a halogen, such as iodine.
In one embodiment the strong base is tert-butyl lithium or similar.
In one embodiment the palladium catalyst is tetrakis(triphenylphosphine)palladium.
The intermediate nitro compound wherein X is NR⁷ can be prepared by the following generic route:

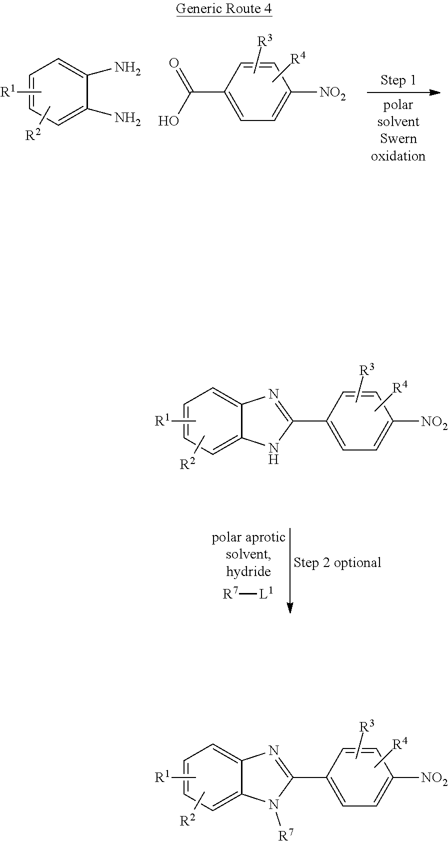

wherein
R¹, R², R³, R⁴ and R⁷ are defined above for compounds of formula (I), and L¹ is a leaving group, with the proviso that R⁷ is other than hydrogen.

In one embodiment step 1 employs the polar solvent DCM (dichloromethane) and a small amount of DMF (dimethyl formamide).

The Swern oxidation generally employs oxalyl chloride and an organic base, such as triethylamine.

In one embodiment the polar aprotic solvent employed in step 2 is DMF.

In one embodiment the hydride employed in step 2 is sodium hydride.

In one embodiment the leaving group L¹ is a halogen, such as iodine.

Other details of the process condition can be seen in the examples given herein.

Protecting groups may be required to protect chemically sensitive groups during one or more of the reactions described above, to ensure that the process is efficient. Thus, if desired or necessary, intermediate compounds may be protected by the use of conventional protecting groups. Protecting groups and means for their removal are described in “Protective Groups in Organic Synthesis”, by Theodora W. Greene and Peter G. M. Wuts, published by John Wiley & Sons Inc; 4^{th} Rev Ed., 2006, ISBN-10: 0471697540.

Examples of salts of compound of the present disclosure include all pharmaceutically acceptable salts, such as, without limitation, acid addition salts of strong mineral acids, such as HCl and HBr salts and addition salts of strong organic acids such as a methanesulfonic acid salt.

The present disclosure extends to solvates of the compounds disclosed herein. Examples of solvates include hydrates.

Novel intermediates are an aspect of the invention.

Also provided herein is a pharmaceutically composition comprising a compound according to the present disclosure and an excipient diluent or carrier. A thorough discussion of pharmaceutically acceptable carriers is available in Remington's Pharmaceutical Sciences (Mack Publishing Company, N.J. 1991).

The pharmaceutical compositions of this disclosure may be administered by any number of routes including, but not limited to, oral, intravenous, intramuscular, intra-arterial, intramedullary, intrathecal, intraventricular, transdermal, transcutaneous (for example, see WO98/20734), subcutaneous, intraperitoneal, intranasal, enteral, topical, sublingual, intravaginal or rectal routes. Hyposprays may also be used to administer the pharmaceutical compositions of the invention.

In one embodiment the therapeutic compositions may be prepared as injectables, either as liquid solutions or suspensions. Solid forms suitable for solution in, or suspension in, liquid vehicles prior to injection may also be prepared.

In one embodiment the pharmaceutical composition according to the present disclosure is provided as a tablet or capsules for oral administration.

### Treatment

The present disclosure also extends to methods of treating a patient comprising administering a therapeutically effective amount of a compound of the present disclosure (or a pharmaceutical composition comprising the same), for example for the treatment of cancer.

Also provide is a compound according to the present disclosure (or a pharmaceutical composition comprising the same) for use in treatment, for example for use in the treatment of cancer.

In a further aspect there is provided a compound of the present disclosure (or a pharmaceutical composition comprising the same) for use in the manufacture of a medicament for the treatment of cancer.

In one embodiment the cancer is an epithelial cancer, for example selected from example is selected from liver cancer (such as hepatocellular carcinoma), biliary tract cancer, breast cancer (such as none ER+ breast cancer), prostate cancer, colorectal cancer, ovarian cancer, cervical cancer, lung cancer, gastric cancer, pancreatic, bone cancer, bladder cancer, head and neck cancer, thyroid cancer, skin cancer, renal cancer, and oesophagus cancer, for example gastric cancer.

In one embodiment the cancer is selected from selected from the group comprising hepatocellular carcinoma, cholangiocarcinoma, breast cancer, prostate cancer, colorecetal cancer, ovarian cancer, lung cancer, gastric cancer, pancreatic and oesophagus cancer.

In one embodiment the biliary duct cancer is in a location selected from intrahepatic bile ducts, left hepatic duct, right hepatic duct, common hepatic duct, cystic duct, common bile duct, Ampulla of Vater and combinations thereof.

In one embodiment the biliary duct cancer is in an intrahepatic bile duct. In one embodiment the biliary duct cancer is in a left hepatic duct. In one embodiment the biliary duct cancer is in a right hepatic duct. In one embodiment the biliary duct cancer is in a common hepatic duct. In one embodiment the biliary duct cancer is in a cystic duct. In one embodiment the biliary duct cancer is in a common bile duct. In one embodiment the biliary duct cancer is in an Ampulla of Vater. In one embodiment the epithelial cancer is a carcinoma.

In one embodiment the treatment according to the disclosure is adjuvant therapy, for example after surgery.

In one embodiment the therapy according to the disclosure is neoadjuvant treatment, for example to shrink a tumour before surgery.

In one embodiment the tumour is a solid tumour. In one embodiment the cancer is a primary cancer, secondary cancer, metastasis or combination thereof. In one embodiment the treatment according to the present disclosure is suitable for the treatment of secondary tumours. In one embodiment the cancer is metastatic cancer. In one embodiment the treatment according to the present disclosure is suitable for the treatment of primary cancer and metastases. In one embodiment the treatment according to the present disclosure is suitable for the treatment of secondary cancer and metastases. In one embodiment the treatment according to the present disclosure is suitable for the treatment of primary cancer, secondary cancer and metastases.

In one embodiment the treatment according to the present disclosure is suitable for the treatment of cancerous cells in a lymph node.

In one embodiment the liver cancer is primary liver cancer. In one embodiment the liver cancer is secondary liver cancer. In one embodiment the liver cancer is stage 1, 2, 3A, 3B, 3C, 4A or 4B.

In one embodiment the gastric cancer is stage 0, I, II, III or IV.

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. This amount can be determined by routine experimentation and is within the judgement of the clinician. Generally, a therapeutically effective amount will be from 0.01 mg/kg to 1000 mg/kg, for example 0.1 mg/kg to 500 mg/kg. Pharmaceutical compositions may be conveniently presented in unit dose forms containing a predetermined amount of an active agent of the invention per dose.

### Combination Therapy

In one embodiment the compound of the present disclosure is employed in combination therapy, for example wherein the further therapy is an anticancer therapy.

In one embodiment the anticancer therapy is a chemotherapy.

Chemotherapeutic agent and chemotherapy or cytotoxic agent are employed interchangeably herein unless the context indicates otherwise.

Chemotherapy as employed herein is intended to refer to specific antineoplastic chemical agents or drugs that are “selectively” destructive to malignant cells and tissues, for example alkylating agents, antimetabolites including thymidylate synthase inhibitors, anthracyclines, anti-microtubule agents including plant alkaloids, topoisomerase inhibitors, parp inhibitors and other anti-tumour agents. Selectively in this context is used loosely because of course many of these agents have serious side effects.

The preferred dose may be chosen by the practitioner, based on the nature of the cancer being treated.

Examples of alkylating agents, which may be employed in the method of the present disclosure include an alkylating agent nitrogen mustards, nitrosoureas, tetrazines, aziridines, platins and derivatives, and non-classical alkylating agents.

Examples of a platinum containing chemotherapeutic agent (also referred to as platins), include cisplatin, carboplatin, oxaliplatin, satraplatin, picoplatin, nedaplatin, triplatin and lipoplatin (a liposomal version of cisplatin), in particular cisplatin, carboplatin and oxaliplatin.

The dose for cisplatin ranges from about 20 to about 270 mg/m² depending on the exact cancer. Often the dose is in the range about 70 to about 100 mg/m².

Nitrogen mustards include mechlorethamine, cyclophosphamide, melphalan, chlorambucil, ifosfamide and busulfan.

Nitrosoureas include N-Nitroso-N-methylurea (MNU), carmustine (BCNU), lomustine (CCNU) and semustine (MeCCNU), fotemustine and streptozotocin. Tetrazines include dacarbazine, mitozolomide and temozolomide.

Aziridines include thiotepa, mytomycin and diaziquone (AZQ).

Examples of antimetabolites, which may be employed in the method of the present disclosure, include anti-folates (for example methotrexate and pemetrexed), purine analogues (for example thiopurines, such as azathiopurine, mercaptopurine, thiopurine, fludarabine (including the phosphate form), pentostatin and cladribine), pyrimidine analogues (for example fluoropyrimidines, such as 5-fluorouracil and prodrugs thereof such as capecitabine [Xeloda®]), floxuridine, gemcitabine, cytarabine, decitabine, raltitrexed (tomudex) hydrochloride, cladribine and 6-azauracil.

Examples of anthracyclines, which may be employed in the method of the present disclosure, include daunorubicin (Daunomycin), daunorubicin (liposomal), doxorubicin (Adriamycin), doxorubicin (liposomal), epirubicin, idarubicin, valrubicin currently used only to treat bladder cancer and mitoxantrone an anthracycline analog, in particular doxorubicin.

Examples of anti-microtubule agents, which may be employed in the method of the present disclosure, include vinca alkaloids and taxanes.

Vinca alkaloids include completely natural chemicals for example vincristine and vinblastine and also semi-synthetic vinca alkaloids, for example vinorelbine, vindesine, and vinflunine Taxanes include paclitaxel, docetaxel, abraxane, carbazitaxel and derivatives of thereof.

Derivatives of taxanes as employed herein includes reformulations of taxanes like taxol, for example in a micelluar formulations, derivatives also include chemical derivatives wherein synthetic chemistry is employed to modify a starting material which is a taxane.

Topoisomerase inhibitors, which may be employed in a method of the present disclosure include type I topoisomerase inhibitors, type II topoisomerase inhibitors and type II topoisomerase poisons. Type I inhibitors include topotecan, irinotecan, indotecan and indimitecan. Type II inhibitors include genistein and ICRF 193 which has the following structure:

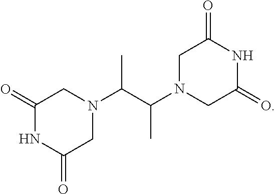 [SMILES: CC(C(C)N1CC(=O)NC(=O)C1)N1CC(=O)NC(=O)C1]

Type II poisons include amsacrine, etoposide, etoposide phosphate, teniposide and doxorubicin and fluoroquinolones.

In one embodiment a combination of chemotherapeutic agents employed is, for example a platin and 5-FU or a prodrug thereof, for example cisplatin or oxaplatin and capecitabine or gemcitabine, such as FOLFOX.

In one embodiment the chemotherapy comprises a combination of chemotherapy agents, in particular cytotoxic chemotherapeutic agents.

In one embodiment the chemotherapy combination comprises a platin, such as cisplatin and fluorouracil or capecitabine.

In one embodiment the chemotherapy combination in capecitabine and oxaliplatin (Xelox).

In one embodiment the chemotherapy is a combination of folinic acid and 5-FU, optionally in combination with oxaliplatin.

In one embodiment the chemotherapy is a combination of folinic acid, 5-FU and irinotecan (FOLFIRI), optionally in combination with oxaliplatin (FOLFIRINOX). The regimen consists of: irinotecan (180 mg/m² IV over 90 minutes) concurrently with folinic acid (400 mg/m² [or 2×250 mg/m²] IV over 120 minutes); followed by fluorouracil (400-500 mg/m² IV bolus) then fluorouracil (2400-3000 mg/m² intravenous infusion over 46 hours). This cycle is typically repeated every two weeks. The dosages shown above may vary from cycle to cycle.

In one embodiment the chemotherapy combination employs a microtubule inhibitor, for example vincristine sulphate, epothilone A, N-[2-[(4-Hydroxyphenyl)amino]-3-pyridinyl]-4-methoxybenzenesulfonamide (ABT-751), a taxol derived chemotherapeutic agent, for example paclitaxel, abraxane, or docetaxel or a combination thereof.

In one embodiment the chemotherapy combination comprises an antimetabolite, such as capecitabine (xeloda), fludarabine phosphate, fludarabine (fludara), decitabine, raltitrexed (tomudex), gemcitabine hydrochloride and cladribine.

In one embodiment the anticancer therapy combination employs an mTor inhibitor. Examples of mTor inhibitors include: everolimus (RAD001), WYE-354, KU-0063794, papamycin (Sirolimus), Temsirolimus, Deforolimus (MK-8669), AZD8055 and BEZ235(NVP-BEZ235).

In one embodiment the anticancer therapy combination employs a MEK inhibitor. Examples of MEK inhibitors include: AS703026, CI-1040 (PD184352), AZD6244 (Selumetinib), PD318088, PD0325901, AZD8330, PD98059, U0126-EtOH, BIX 02189 or BIX 02188.

In one embodiment the chemotherapy combination employs an AKT inhibitor. Examples of AKT inhibitors include: MK-2206 and AT7867.

In one embodiment the anticancer therapy employs an aurora kinase inhibitor. Examples of aurora kinase inhibitors include: Aurora A Inhibitor I, VX-680, AZD1152-HQPA (Barasertib), SNS-314 Mesylate, PHA-680632, ZM-447439, CCT129202 and Hesperadin.

In one embodiment the chemotherapy combination employs a p38 inhibitor, for example as disclosed in WO2010/038086, such as N-[4-({4-[3-(3-tert-Butyl-1-p-tolyl-1H-pyrazol-5-yl)ureido]naphthalen-1-yloxy}methyl)pyridin-2-yl]-2-methoxyacetamide.

In one embodiment the combination employs a Bcl-2 inhibitor. Examples of Bcl-2 inhibitors include: obatoclax mesylate, ABT-737, ABT-263(navitoclax) and TW-37.

In one embodiment the chemotherapy combination comprises ganciclovir, which may assist in controlling immune responses and/or tumour vasculation.

In one embodiment the anticancer therapy includes a PARP inhibitor.

In one embodiment the anticancer therapy includes an inhibitor of cancer metabolism with specific inhibition of the activity of the DHODH enzyme.

In one embodiment one or more therapies employed in the method herein are metronomic, that is a continuous or frequent treatment with low doses of anticancer drugs, often given concomitant with other methods of therapy.

In one embodiment, there is provided the use of multiple cycles of treatment (such as chemotherapy) for example 2, 3, 4, 5, 6, 7, 8.

Comprising” in the context of the present specification is intended to mean “including”. Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

The present application claims priority from SG 10201807515T filed 31 Aug. 2018, incorporated herein by reference. The priority document may be used as the basis for corrections.

The invention will now be described with reference to the following examples, which are merely illustrative and should not in any way be construed as limiting the scope of the present invention.

### EXAMPLES

### General Method 1 methyl benzo[d]oxazole-7-carboxylate

A solution of methyl 3-amino-2-hydroxybenzoate (1 g, 5.99 mmol) and p-toluenesulfonic acid (0.1 g, 0.581 mmol) in trimethylorthoformate (50 mL) was refluxed for 1 hour. Volatiles were evaporated under reduced pressure. The crude was purified by column chromatography on silica gel using 20:1 DCM:EtOAc as eluent to give methyl benzo[d]oxazole-7-carboxylate (0.99 g, 93%). UPLC-MS (Acidic Method, 2 min): rt 0.79 min, m/z 178.1 [M+H]⁺

### General Method 2 methyl 2-(4-nitrophenyl)benzo[d]oxazole-7-carboxylate

A solution of methyl benzo[d]oxazole-7-carboxylate (0.5 g, 2.82 mmol), 1-iodo-4-nitrobenzene (0.88 g, 3.53 mmol), lithium tert-butoxide (0.45 g, 5.64 mmol), tetrakis(triphenyl phosphine)palladium(0) (0.16 g, 0.141 mmol) in dioxane under a nitrogen atmosphere was stirred at room temperature for 45 minutes. The resulting thick suspension was diluted with EtOAc, washed with water, organics washed with brine, dried over Na₂SO₄, decanted and solvent removed under vacuum. The crude was purified by column chromatography on silica gel using 20:1 to 5:1 Hexane:EtOAc as eluent to give methyl 2-(4-nitrophenyl)benzo[d]oxazole-7-carboxylate (0.4 g, 48%). UPLC-MS (Acidic Method, 2 min): rt 1.11 min, m/z 299.0 [M+H]⁺

### General Method 3 2-(4-aminophenyl)-N-methylbenzo[d]oxazole-7-carboxamide

To a suspension of N-methyl-2-(4-nitrophenyl)benzo[d]oxazole-7-carboxamide (110 mg, 0.37 mmol) in MeOH (5 mL) was added 10% Pd/C in a MeOH slurry and the mixture was stirred at room temperature for 18 hours under a hydrogen atmosphere (balloon). The reaction mixture was filtered through celite and the filtrate volume was reduced to dryness under vacuum to give 2-(4-aminophenyl)-N-methylbenzo[d]oxazole-7-carboxamide (85 mg, 86%). UPLC-MS (Acidic Method, 2 min): rt 0.79 min, m/z 268.1 [M+H]⁺

### General Method 4 2-(2-methyl-4-nitrophenyl)benzo[d]oxazole

To a solution of 2-methyl-4-nitrobenzoic acid (1.66 g, 9.17 mmol) in DCM (15 mL) containing 5 drops of DMF was added oxalyl chloride (1.17 mL, 13.8 mmol) drop wise and the suspension was stirred at room temperature for 2 hours to form a clear solution. Volatiles were removed under vacuum and to the resulting crude acid chloride, was added a suspension of 2-aminophenol (1 g, 9.17 mmol) and triethylamine (3.3 mL, 22.9 mmol) in DCM (15 mL), drop wise, while vigorously stirring, and the reaction was continued at ambient temperature for 72 hours. The obtained suspension was filtered, and the filtrate volume was reduced to dryness under vacuum, taken up in dioxane (20 mL) and refluxed for 48 hours after adding methylsulfonic acid (1.8 mL, 27.6 mmol) to it. The reaction mixture was cooled down to room temperature, diluted with DCM, washed with saturated sodium bicarbonate, brine, and the organic liquor was dried over Na₂SO₄, decanted and evaporated under reduced pressure. The crude was purified by column chromatography on silica gel using 5:1 Hexane:EtOAc as mobile phase to give 2-(2-methyl-4-nitrophenyl)benzo[d]oxazole (0.72 g, 31%). UPLC-MS (Acidic Method, 2 min): rt 1.30 min, m/z 255.1 [M+H]⁺

### General Method 5 N-(4-(benzo[d]oxazol-2-yl)-3-methylphenyl)isoxazole-3-carboxamide

A solution of 1-methyl-1H-pyrazole-5-carboxylic acid (28 mg, 0.223 mmol), HATU (84 mg, 0.223 mmol), triethylamine (95 ul, 0.67 mmol) and catalytic amount of DMAP in THF (2 mL) was stirred at room temperature for 5 minutes. 4-(benzo[d]oxazol-2-yl)-3-methylaniline (50 mg, 0.223 mmol) was added and the resulting mixture was stirred at 65° C. for 18 h. The reaction mixture was diluted with DCM, washed with saturated solution of NaHCO₃ and brine. The organic solution was, dried over Na₂SO₄, decanted and evaporated under reduced pressure. The crude was purified by column chromatography on silica gel using 1:4 to 1:2 EtOAc:Hexane as mobile phase to give N-(4-(benzo[d]oxazol-2-yl)-3-methylphenyl)isoxazole-3-carboxamide (18 mg, 24%). UPLC-MS (Acidic Method, 4 min): rt 1.96 min, m/z 333.1 [M+H]⁺

### Example 1 Preparation of N-(4-(1-ethyl-1H-benzo[d]imidazol-2-yl)phenyl)-1-methyl-1H-pyrazole-5-carboxamide

The preparation is summarised in scheme 1 below:

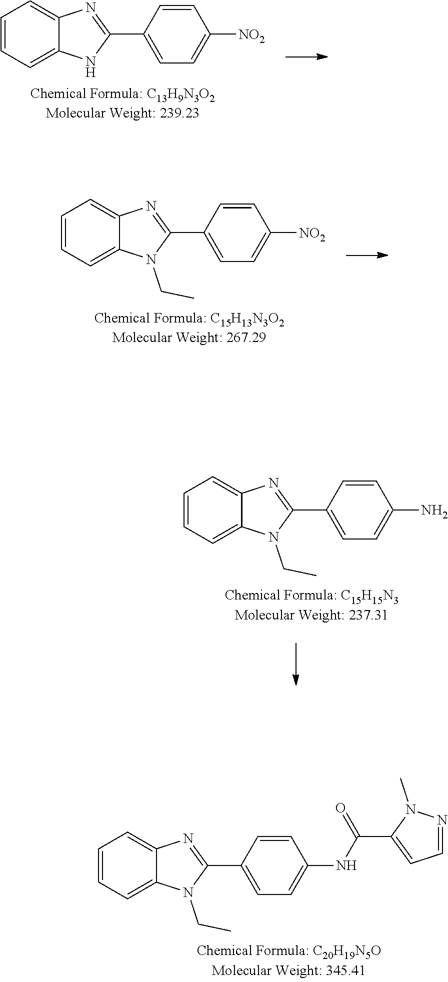 [SMILES: O=[N+]([O-])c1ccc(-c2nc3ccccc3[nH]2)cc1 ; CCn1c(-c2ccc([N+](=O)[O-])cc2)nc2ccccc21 ; CCn1c(-c2ccc(N)cc2)nc2ccccc21 ; CCn1c(-c2ccc(NC(=O)c3ccnn3C)cc2)nc2ccccc21]

### 1-Ethyl-2-(4-nitrophenyl)-1H-benzo[d]imidazole

A solution of 2-(4-Nitrophenyl)-1H-benzo[d]imidazole (200 mg, 0.84 mmol) and sodium hydride (1.1 equiv., 0.92 mmol) in DMF (3 mL) was stirred at room temperature for 30 mins. Iodoethane (1.5 equiv., 1.25 mmol) was added and the resulting mixture was stirred at 60° C. for 18h. The reaction mixture was diluted in water and extracted twice with EtOAc. The organic solution was washed with brine solution, dried over Na₂SO₄ and evaporated under reduced pressure. The crude was purified by column chromatography on silica gel using ethyl acetate in hexane as mobile phase to give 1-Ethyl-2-(4-nitrophenyl)-1H-benzo[d]imidazole (0.18 g, 80%) as a yellowish solid. UPLC-MS (Acidic Method, 2 min): rt 0.86 min, m/z 268.2 [M+H]⁺

¹H NMR (400 MHz, DMSO-d₆) δ ppm: 8.42 (d, J=9.0 Hz, 2H), 8.07-8.12 (m, 2H), 7.70-7.76 (m, 2H), 7.27-7.39 (m, 2H), 4.35-4.42 (m, 2H), 1.36 (t, J=7.2 Hz, 3H)

### 4-(1-Ethyl-1H-benzo[d]imidazol-2-yl)aniline

To a solution of 1-Ethyl-2-(4-nitrophenyl)-1H-benzo[d]imidazole (180 mg, 0.67 mmol) in ethanol (7 mL) under N₂ was added Pd/C (50% w/w). The resulting mixture was stirred at room temperature under H₂ for 18 h. The reaction mixture was filtrated on celite pad and rinsed with ethanol. The filtrate was evaporated under reduced pressure to give 4-(1-Ethyl-1H-benzo[d]imidazol-2-yl)aniline (0.21 g, 88% purity) as crude compound (yellow oil). The crude compound was taken to the next step without further purification. UPLC-MS (Basic Method, 2 min): rt 0.87 min, m/z 238.2 [M+H]⁺

### N-(4-(1-ethyl-1H-benzo[d]imidazol-2-yl)phenyl)-1-methyl-1H-pyrazole-5-carboxamide

A suspension of 1-methyl-1H-pyrazole-5-carboxylic acid (112 mg, 0.886 mmol), HATU (1.0 equiv., 0.886 mmol), triethylamine (3.0 equiv., 2.66 mmol), 4-(1-ethyl-1H-benzo[d]imidazol-2-yl)aniline (1.0 equiv., 0.886) and catalytic amount of DMAP in THF (8 mL) was stirred at 70° C. for 18 h. The reaction mixture was diluted with DCM, washed with saturated solution of NaHCO₃ and brine. The organic solution was, dried over Na₂SO₄, decanted and evaporated under reduced pressure. The crude was purified by column chromatography on silica gel using 1:1 to 3:1 EtOAc:Hexane as mobile phase. The resulting product was triturated in EtOAc/Hexane, filtered and dried to give N-(4-(1-ethyl-1H-benzo[d]imidazol-2-yl)phenyl)-1-methyl-1H-pyrazole-5-carboxamide (26 mg, 9%) as a white solid. UPLC-MS (Basic Method, 4 min): rt 1.59 min, m/z 346.1 [M+H]⁺

¹H NMR (400 MHz, DMSO-d₆) δ: 10.45 (s, 1H), 7.93-7.97 (m, 2H), 7.77-7.82 (m, 2H), 7.63-7.69 (m, 2H), 7.57 (d, J=2.1 Hz, 1H), 7.19-7.31 (m, 2H), 7.11 (d, J=2.0 Hz, 1H), 4.34 (q, J=7.2 Hz, 2H), 4.11 (s, 3H), 1.35 (t, J=7.2 Hz, 3H)

### Example 2 Preparation of N-(4-(benzo[d]oxazol-2-yl)phenyl)-1-(2-methoxyethyl)-1H-pyrazole-5-carboxamide

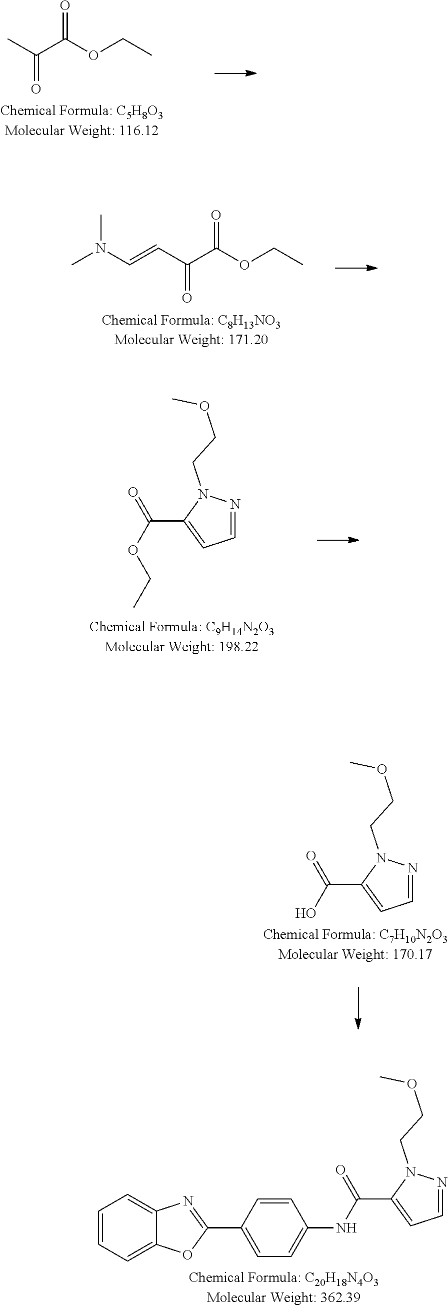

### Ethyl (E)-4-(dimethylamino)-2-oxobut-3-enoate

A solution of ethyl 2-oxopropanoate (5.0 g, 43.1 mmol) in N,N-Dimethylformamide dimethyl acetal (1.0 equiv., 43.1 mmol) was stirred at room temperature for 18 h. The solvent was removed under reduced pressure and the crude was purified by column chromatography on silica gel using methanol in DCM as mobile phase to give Ethyl (E)-4-(dimethylamino)-2-oxobut-3-enoate (2.5 g, 34%) as a brown oil. UPLC-MS (Acidic Method, 2 min): rt 0.62 min, m/z 172.1 [M+H]⁺

### Ethyl 1-(2-methoxyethyl)-1H-pyrazole-5-carboxylate

A solution of Ethyl (E)-4-(dimethylamino)-2-oxobut-3-enoate (1.06 g, 6.2 mmol) and (2-methoxyethyl)hydrazine (1.0 equiv., 6.2 mmol) in ethanol was stirred at 60° C. for 18 h. The solvent was evaporated under reduced pressure and the crude was purified by column chromatography on silica gel using methanol in DCM as mobile phase to give Ethyl 1-(2-methoxyethyl)-1H-pyrazole-5-carboxylate (305 mg, 25%) as a pale yellow oil. UPLC-MS (Acidic Method, 2 min): rt 0.83 min, m/z 199.2 [M+H]⁺

¹H NMR (400 MHz, DMSO-d₆) δ ppm: 7.55-7.59 (m, 1H), 6.86 (d, J=2.0 Hz, 1H), 4.63-4.70 (m, 2H), 4.29 (d, J=7.2 Hz, 2H), 3.67 (s, 2H), 3.18 (s, 3H), 1.30 (t, J=7.1 Hz, 3H)

### 1-(2-Methoxyethyl)-1H-pyrazole-5-carboxylic acid

To a solution of Ethyl 1-(2-methoxyethyl)-1H-pyrazole-5-carboxylate (150 mg, 0.76 mmol) in ethanol was added a solution of NaOH (1.1 equiv., 0.84 mmol) in water (1 mL). The resulting mixture was stirred at room temperature for 1.5 h. Ethanol was removed under vacuum, the residue was acidified with a solution of 1M HCl, and the resulting aqueous solution was extracted with DCM. The organic solution was dried over Na₂SO₄ and evaporated under reduced pressure to give 1-(2-Methoxyethyl)-1H-pyrazole-5-carboxylic acid (105 mg, 81%) as a white solid. UPLC-MS (Acidic Method, 2 min): rt 0.54 min, m/z 171.1 [M+H]⁺

¹H NMR (400 MHz, CDCl₃) δ ppm: 7.56-7.58 (m, 1H), 6.95-6.97 (m, 1H), 4.76-4.81 (m, 2H), 3.79-3.83 (m, 2H), 3.34-3.36 (m, 3H), 0.08 (s, 1H)

### N-(4-(benzo[d]oxazol-2-yl)phenyl)-1-(2-methoxyethyl)-1H-pyrazole-5-carboxamide

A solution of 1-(2-Methoxyethyl)-1H-pyrazole-5-carboxylic acid (100 mg, 0.58 mmol), HATU (2.0 equiv., 1.17 mmol), triethylamine (6.0 equiv., 3.5 mmol) and catalytic amount of DMAP in THF was stirred at room temperature for 10 mins. 4-(benzo[d]oxazol-2-yl)aniline (1.5 equiv. 0.88 mmol) was added and the resulting mixture was stirred at 70° C. for 72 h. The reaction mixture was diluted in water and DCM then the aqueous solution was extracted twice with DCM. The organic solution was washed with saturated solution of NaHCO₃ and brine, dried over Na₂SO₄ and evaporated under reduced pressure. The crude was purified by column chromatography on silica gel using 50% ethyl acetate in hexane as mobile phase. The resulting product was triturated in hexane/Et₂O, filtrated, washed with Et₂O and dried to give N-(4-(benzo[d]oxazol-2-yl)phenyl)-1-(2-methoxyethyl)-1H-pyrazole-5-carboxamide (26 mg, 12%) as a white solid. UPLC-MS (Acidic Method, 2 min): rt 1.05 min, m/z 363.1 [M+H]⁺

¹H NMR (400 MHz, DMSO-d₆) δ ppm: 10.58 (s, 1H), 8.19-8.24 (m, 2H), 7.97-8.02 (m, 2H), 7.75-7.82 (m, 2H), 7.61 (d, J=2.0 Hz, 1H), 7.38-7.45 (m, 2H), 7.08 (d, J=2.0 Hz, 1H), 4.70 (t, J=5.6 Hz, 2H), 3.69 (t, J=5.6 Hz, 2H), 3.19 (s, 3H), 2.69 (s, 1H)

### Example 3 Preparation of methyl 2-(4-(1-methyl-1H-pyrazole-5-carboxamido)phenyl) benzo[d]oxazole-7-carboxylate

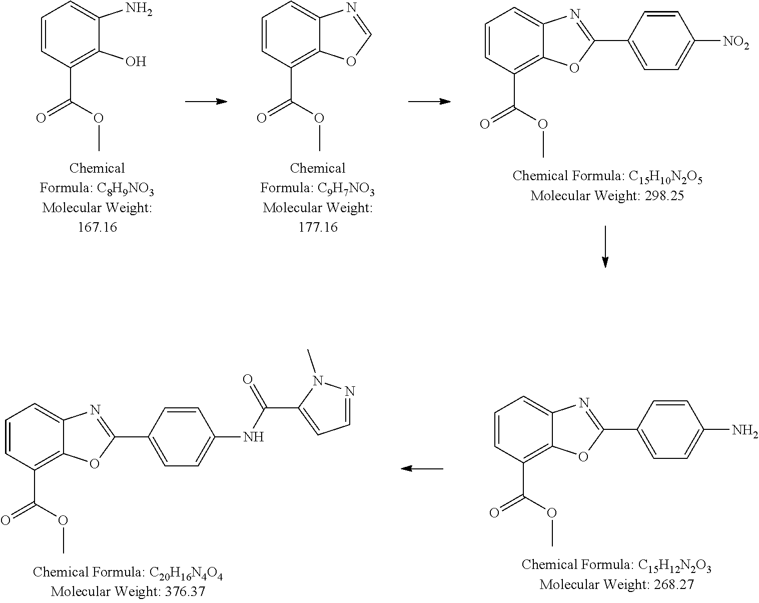 [SMILES: COC(=O)c1cccc(N)c1O ; COC(=O)c1cccc2ncoc12 ; COC(=O)c1cccc2nc(-c3ccc([N+](=O)[O-])cc3)oc12 ; COC(=O)c1cccc2nc(-c3ccc(NC(=O)c4ccnn4C)cc3)oc12 ; COC(=O)c1cccc2nc(-c3ccc(N)cc3)oc12]

### Methyl benzo[d]oxazole-7-carboxylate

Compound prepared according to general METHOD 1 to give methyl benzo[d]oxazole-7-carboxylate. UPLC-MS (Acidic Method, 2 min): rt 0.86 min, m/z 178.0 [M+H]⁺

### Methyl 2-(4-nitrophenyl)benzo[d]oxazole-7-carboxylate

Compound prepared according to general METHOD 2 to give Methyl 2-(4-nitrophenyl)benzo[d]oxazole-7-carboxylate. UPLC-MS (Acidic Method, 2 min): rt 1.19 min, m/z 299.0 [M+H]⁺

### Methyl 2-(4-aminophenyl)benzo[d]oxazole-7-carboxylate

To a suspension of methyl 2-(4-nitrophenyl)benzo[d]oxazole-7-carboxylate (20 mg, 0.067 mmol) in EtOH (1 mL) was added 10% Pd/C and the mixture was stirred at room temperature for 72 hours under a hydrogen atmosphere (balloon). The reaction mixture was filtered through celite and the filtrate volume was reduced to dryness under vacuum to give methyl 2-(4-aminophenyl)benzo[d]oxazole-7-carboxylate (18 mg, 100%). UPLC-MS (Acidic Method, 2 min): rt 1.02 min, m/z 269.1 [M+H]⁺

### Methyl 2-(4-(1-methyl-1H-pyrazole-5-carboxamido)phenyl)benzo[d]oxazole-7-carboxylate

A solution of 1-methyl-1H-pyrazole-5-carboxylic acid (17 mg, 0.134 mmol), HATU (51 mg, 0.134 mmol), triethylamine (41 mg, 0.402 mmol) and catalytic amount of DMAP in THF (1 mL) was stirred at room temperature for 10 minutes. Methyl 2-(4-aminophenyl)benzo[d]oxazole-7-carboxylate (18 mg, 0.067 mmol) was added and the resulting mixture was stirred at 75° C. for 72 h. The reaction mixture was diluted with DCM, washed with saturated solution of NaHCO₃ and brine. The organic solution was, dried over Na₂SO₄, decanted and evaporated under reduced pressure. The crude was purified by column chromatography on silica gel using 1:1 EtOAc:Hexane as mobile phase. The resulting product was triturated in diethyl ether, filtered and dried to give methyl 2-(4-(1-methyl-1H-pyrazole-5-carboxamido)phenyl)benzo[d]oxazole-7-carboxylate (2 mg, 8%). UPLC-MS (Acidic Method, 4 min): rt 1.77 min, m/z 377.1 [M+H]⁺

¹H NMR (400 MHz, CHLOROFORM-d) δ ppm 8.36 (d, J=8.7 Hz, 2H), 7.95-8.03 (m, 2H), 7.83 (br d, J=8.7 Hz, 3H), 7.56 (d, J=1.5 Hz, 1H), 7.45 (s, 1H), 6.72 (s, 1H), 4.24-4.29 (m, 3H), 4.08 (s, 3H)

### Example 4 Preparation of N-methyl-2-(4-(1-methyl-1H-pyrazole-5-carboxamido)phenyl) benzo[d]oxazole-7-carboxamide

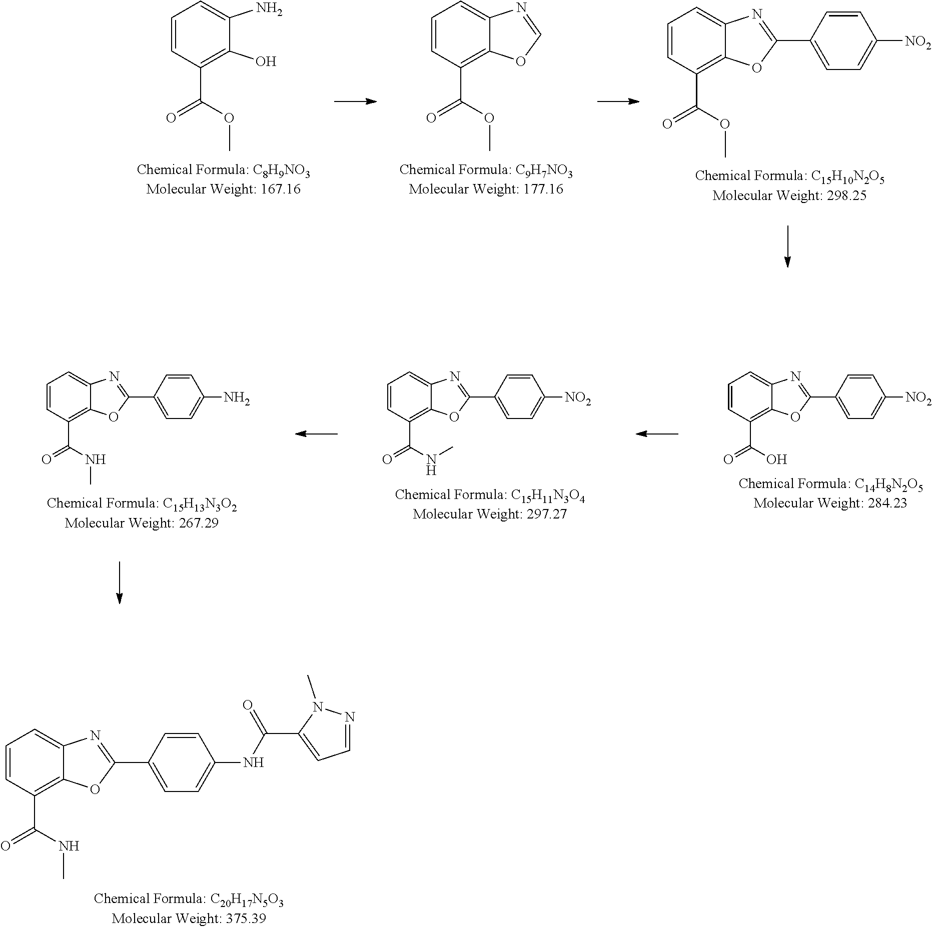

### methyl benzo[d]oxazole-7-carboxylate. (General Method 1)

A solution of methyl 3-amino-2-hydroxybenzoate (1 g, 5.99 mmol) and p-toluenesulfonic acid (0.1 g, 0.581 mmol) in trimethylorthoformate (50 mL) was refluxed for 1 hour. Volatiles were evaporated under reduced pressure. The crude was purified by column chromatography on silica gel using 20:1 DCM:EtOAc as eluent to give methyl benzo[d]oxazole-7-carboxylate (0.99 g, 93%). UPLC-MS (Acidic Method, 2 min): rt 0.79 min, m/z 178.1 [M+H]⁺

### Methyl 2-(4-nitrophenyl)benzo[d]oxazole-7-carboxylate (General Method 2)

A solution of methyl benzo[d]oxazole-7-carboxylate (0.5 g, 2.82 mmol), 1-iodo-4-nitrobenzene (0.88 g, 3.53 mmol), lithium tert-butoxide (0.45 g, 5.64 mmol), tetrakis(triphenylphosphine)palladium(0) (0.16 g, 0.141 mmol) in dioxane under a nitrogen atmosphere was stirred at room temperature for 45 minutes. The resulting thick suspension was diluted with EtOAc, washed with water, organics washed with brine, dried over Na₂SO₄, decanted and solvent removed under vacuum. The crude was purified by column chromatography on silica gel using 20:1 to 5:1 Hexane:EtOAc as eluent to give methyl 2-(4-nitrophenyl)benzo[d]oxazole-7-carboxylate (0.4 g, 48%). UPLC-MS (Acidic Method, 2 min): rt 1.11 min, m/z 299.0 [M+H]⁺

### 2-(4-nitrophenyl)benzo[d]oxazole-7-carboxylic acid

To a suspension of methyl 2-(4-nitrophenyl)benzo[d]oxazole-7-carboxylate (0.81 g, 2.72 mmol) in THF (20 mL) was added LiOH (0.326 g, 13.6 mmol) dissolved in water (5 mL). The mixture was stirred at room temperature for 78 hours. The reaction mixture pH was taken to acid with 3M HCl, the formed suspension was filtered and the filtered solid was washed with hexane, triturated in acetone, filtered and dried to give 2-(4-nitrophenyl)benzo[d]oxazole-7-carboxylic acid (0.74 g, 96%). UPLC-MS (Acidic Method, 2 min): rt 0.95 min, m/z 285.0 [M+H]⁺

### N-methyl-2-(4-nitrophenyl)benzo[d]oxazole-7-carboxamide

To a solution of 2-(4-nitrophenyl)benzo[d]oxazole-7-carboxylic acid (0.64 g, 2.25 mmol) in DMF (35 mL) was added EDCI (0.646 g, 3.38 mmol) and HOBt (0.52 g, 3.38 mmol) and the solution was stirred for 10 minutes before adding monomethyl amine hydrochloride (0.753 g, 11.2 mmol) and triethylamine (2.5 mL, 18 mmol) drop wise and the formed suspension was stirred at room temperature for 18 hours. The reaction mixture was diluted with water (100 mL) and extracted 4 times with EtOAc; the organic phase was washed with saturated bicarbonate and the formed emulsion was filtered and the obtained solid washed with EtOAc and dried to give N-methyl-2-(4-nitrophenyl)benzo[d]oxazole-7-carboxamide (0.59 g, 88%). UPLC-MS (Acidic Method, 2 min): rt 0.94 min, m/z 298.1 [M+H]⁺

¹H NMR (400 MHz, DMSO-d₆) δ: 8.53-8.57 (m, 2H), 8.45-8.51 (m, 3H), 8.04 (dd, J=7.9, 1.1 Hz, 1H), 7.85 (dd, J=7.8, 1.1 Hz, 1H), 7.55 (t, J=7.8 Hz, 1H), 2.94 (d, J=4.6 Hz, 3H)

### 2-(4-aminophenyl)-N-methylbenzo[d]oxazole-7-carboxamide (General Method 3)

To a suspension of N-methyl-2-(4-nitrophenyl)benzo[d]oxazole-7-carboxamide (110 mg, 0.37 mmol) in MeOH (5 mL) was added 10% Pd/C in a MeOH slurry and the mixture was stirred at room temperature for 18 hours under a hydrogen atmosphere (balloon). The reaction mixture was filtered through celite and the filtrate volume was reduced to dryness under vacuum to give 2-(4-aminophenyl)-N-methylbenzo[d]oxazole-7-carboxamide (85 mg, 86%). UPLC-MS (Acidic Method, 2 min): rt 0.79 min, m/z 268.1 [M+H]⁺

### N-methyl-2-(4-(1-methyl-1H-pyrazole-5-carboxamido)phenyl)benzo[d]oxazole-7-carboxamide

A suspension of 1-methyl-1H-pyrazole-5-carboxylic acid (80 mg, 0.637 mmol), HATU (242 mg, 0.637 mmol), triethylamine (270 ul, 1.9 mmol), 2-(4-aminophenyl)-N-methylbenzo[d]oxazole-7-carboxamide (85 mg, 0.318) and catalytic amount of DMAP in THF (3 mL) was stirred at 70° C. for 18 hours. The reaction mixture was diluted with DCM, washed with saturated solution of NaHCO₃ and brine. The organic solution was, dried over Na₂SO₄, decanted and evaporated under reduced pressure. The crude was purified by column chromatography on silica gel using 1:1 to 9:1 EtOAc:Hexane as mobile phase. The resulting product was triturated in EtOAc, filtered, washed with diethyl ether and dried to give N-methyl-2-(4-(1-methyl-1H-pyrazole-5-carboxamido)phenyl)benzo[d]oxazole-7-carboxamide (16 mg, 13%). UPLC-MS (Acidic Method, 4 min): rt 1.35 min, m/z 376.1 [M+H]⁺

¹H NMR (400 MHz, DMSO-d₆) δ: 10.55 (s, 1H), 8.38 (br d, J=4.3 Hz, 1H), 8.27-8.34 (m, 2H), 8.00-8.05 (m, 2H), 7.93 (dd, J=7.9, 1.1 Hz, 1H), 7.76 (dd, J=7.8, 1.1 Hz, 1H), 7.57 (d, J=2.0 Hz, 1H), 7.48 (t, J=7.8 Hz, 1H), 7.13 (d, J=2.1 Hz, 1H), 4.12 (s, 3H), 2.94 (d, J=4.6 Hz, 3H)

### Example 5 Preparation of N-(4-(4,5-dimethyloxazol-2-yl)phenyl)-1-methyl-1H-pyrazole-5-carboxamide

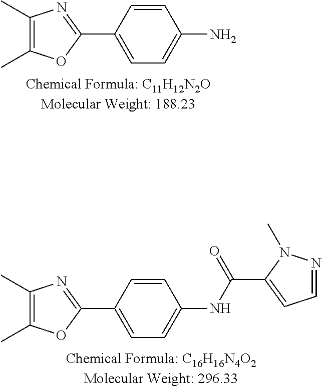 [SMILES: Cc1nc(-c2ccc(N)cc2)oc1C ; Cc1nc(-c2ccc(NC(=O)c3ccnn3C)cc2)oc1C]

A solution of 1-methyl-1H-pyrazole-5-carboxylic acid (2.0 equiv., 1.06 mmol) and HATU (2.0 equiv., 1.06 mmol) in THF was stirred at room temperature for 30 mins. To the reaction mixture was added 4-(4,5-dimethyloxazol-2-yl)aniline (100 mg, 0.53 mmol), followed by trimethylamine (6.0 equiv., 3.19 mmol) and catalytic amount of DMAP, and the resulting mixture was stirred at room temperature for 30 mins. The reaction mixture was diluted in DCM and washed with saturated solution of NaHCO₃ and brine. The organic solution was dried over Na₂SO₄ and evaporated under reduced pressure. The crude was purified by column chromatography on silica gel using 50% ethyl acetate in hexane as mobile phase. The resulting product was triturated in EtOAc, filtrated, washed with Et₂O and dried to give N-(4-(4,5-dimethyloxazol-2-yl)phenyl)-1-methyl-1H-pyrazole-5-carboxamide (62 mg, 40%) as a white powder. UPLC-MS (Acidic Method, 4 min): rt 1.46 min, m/z 297.1 [M+H]⁺

¹H NMR (400 MHz, DMSO-d₆) δ ppm 10.40 (s, 1H), 7.88 (d, J=1.3 Hz, 4H), 7.55 (d, J=2.1 Hz, 1H), 7.09 (d, J=2.0 Hz, 1H), 4.06-4.11 (m, 3H), 2.31 (d, J=0.8 Hz, 3H), 2.06-2.10 (m, 3H)

### Example 6 Preparation of 1-methyl-N-(4-(7-methylbenzo[d]oxazol-2-yl)phenyl)-1H-pyrazole-5-carboxamide

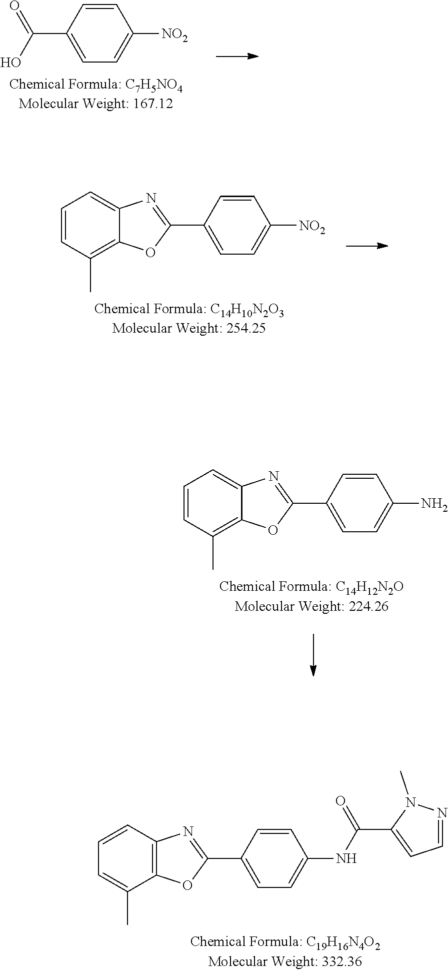 [SMILES: O=C(O)c1ccc([N+](=O)[O-])cc1 ; Cc1cccc2nc(-c3ccc([N+](=O)[O-])cc3)oc12 ; Cc1cccc2nc(-c3ccc(N)cc3)oc12 ; Cc1cccc2nc(-c3ccc(NC(=O)c4ccnn4C)cc3)oc12]

### 7-methyl-2-(4-nitrophenyl)benzo[d]oxazole

Compound prepared according to general METHOD 4 to give 7-methyl-2-(4-nitrophenyl)benzo[d]oxazole. UPLC-MS (Acidic Method, 2 min): rt 1.29 min, m/z 255.1 [M+H]⁺

### 4-(7-methylbenzo[d]oxazol-2-yl)aniline

Compound prepared according to general METHOD 3 (overnight reaction) to give 4-(7-methylbenzo[d]oxazol-2-yl)aniline. UPLC-MS (Acidic Method, 4 min): rt 1.76 min, m/z 225.1 [M+H]⁺ ¹H NMR (400 MHz, DMSO-d₆) δ ppm 7.85-7.89 (m, 2H), 7.46 (d, J=7.8 Hz, 1H), 7.20 (t, J=7.7 Hz, 1H), 7.09-7.14 (m, 1H), 6.67-6.72 (m, 2H), 5.95 (s, 2H), 2.51-2.53 (m, 3H)

### 1-methyl-N-(4-(7-methylbenzo[d]oxazol-2-yl)phenyl)-1H-pyrazole-5-carboxamide

Compound prepared according to general METHOD 5 to give 1-methyl-N-(4-(7-methylbenzo[d]oxazol-2-yl)phenyl)-1H-pyrazole-5-carboxamide. UPLC-MS (Acidic Method, 2 min): rt 1.96 min, m/z 333.1 [M+H]⁺

¹H NMR (400 MHz, DMSO-d₆) δ ppm 10.47-10.55 (m, 1H), 8.19-8.24 (m, 2H), 7.98-8.03 (m, 2H), 7.55-7.61 (m, 2H), 7.26-7.32 (m, 1H), 7.20-7.25 (m, 1H), 7.11-7.15 (m, 1H), 4.08-4.15 (m, 3H), 2.54-2.59 (m, 3H).

### Example 7 N-(4-(benzo[d]oxazol-2-yl)-3-methylphenyl)isoxazole-3-carboxamide

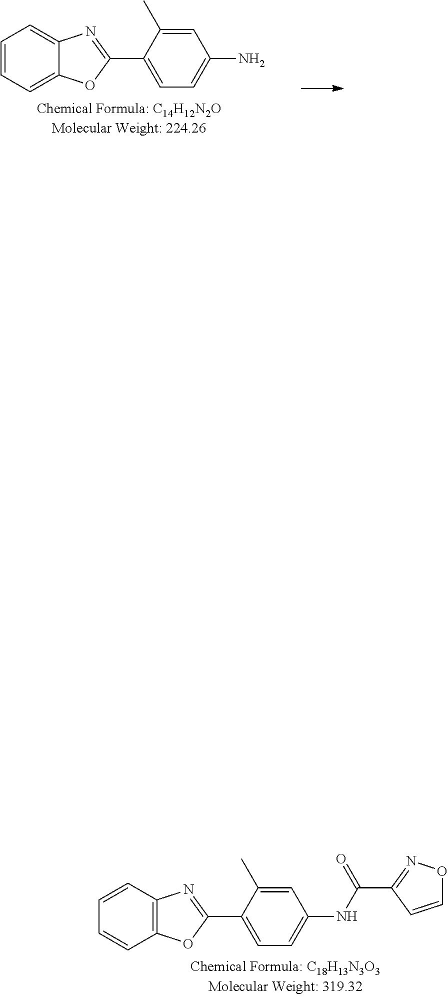 [SMILES: Cc1cc(N)ccc1-c1nc2ccccc2o1 ; Cc1cc(NC(=O)c2ccon2)ccc1-c1nc2ccccc2o1]

### N-(4-(benzo[d]oxazol-2-yl)-3-methylphenyl)isoxazole-3-carboxamide. (General Method 5)

A solution of isoxazole-3-carboxylic acid (28 mg, 0.223 mmol), HATU (84 mg, 0.223 mmol), triethylamine (95 ul, 0.67 mmol) and catalytic amount of DMAP in THF (2 mL) was stirred at room temperature for 5 minutes. 4-(benzo[d]oxazol-2-yl)-3-methylaniline (50 mg, 0.223 mmol) was added and the resulting mixture was stirred at 65° C. for 18 h. The reaction mixture was diluted with DCM, washed with saturated solution of NaHCO₃ and brine. The organic solution was, dried over Na₂SO₄, decanted and evaporated under reduced pressure. The crude was purified by column chromatography on silica gel using 1:4 to 1:2 EtOAc:Hexane as mobile phase to give N-(4-(benzo[d]oxazol-2-yl)-3-methylphenyl)isoxazole-3-carboxamide (18 mg, 24%). UPLC-MS (Acidic Method, 4 min): rt 2.01 min, m/z 320.1 [M+H]⁺

¹H NMR (400 MHz, DMSO-d₆) δ ppm 10.58-11.34 (m, 1H), 8.95-9.42 (m, 1H), 8.16 (br d, J=8.2 Hz, 1H), 7.65-8.01 (m, 4H), 7.27-7.56 (m, 2H), 7.07 (br d, J=1.8 Hz, 1H), 2.67-2.86 (m, 3H)

### Example 8 Preparation of N-(3-methyl-4-(7-methylbenzo[d]oxazol-2-yl)phenyl) isoxazole-3-carboxamide

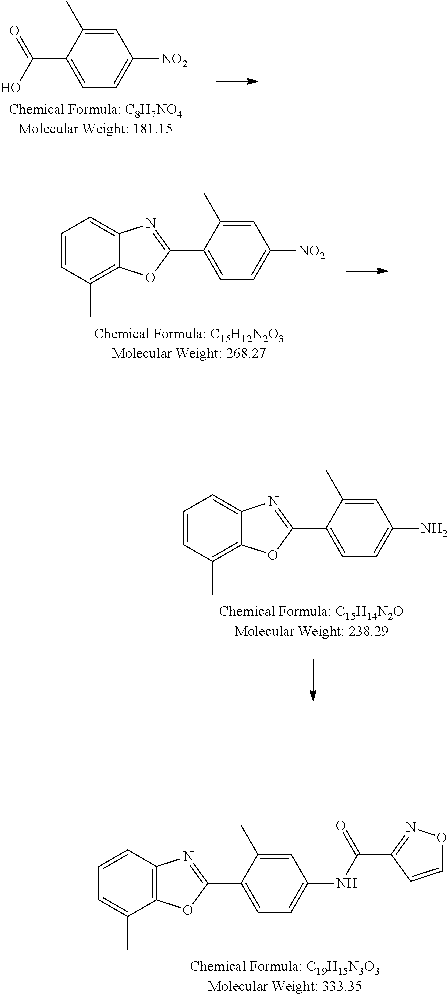 [SMILES: Cc1cc([N+](=O)[O-])ccc1C(=O)O ; Cc1cc([N+](=O)[O-])ccc1-c1nc2cccc(C)c2o1 ; Cc1cc(N)ccc1-c1nc2cccc(C)c2o1 ; Cc1cc(NC(=O)c2ccon2)ccc1-c1nc2cccc(C)c2o1]

### 7-methyl-2-(2-methyl-4-nitrophenyl)benzo[d]oxazole

Compound prepared according to general METHOD 4 to give 7-methyl-2-(2-methyl-4-nitrophenyl)benzo[d]oxazole. UPLC-MS (Acidic Method, 4 min): rt 2.33 min, m/z 269.2 [M+H]⁺

### 3-methyl-4-(7-methylbenzo[d]oxazol-2-yl)aniline

Compound prepared according to general METHOD 3 to give 3-methyl-4-(7-methylbenzo[d]oxazol-2-yl)aniline. UPLC-MS (Acidic Method, 2 min): rt 1.17 min, m/z 239.2 [M+H]⁺

### N-(3-methyl-4-(7-methylbenzo[d]oxazol-2-yl)phenyl)isoxazole-3-carboxamide

A solution of isoxazole-3-carboxylic acid (10 mg, 0.084 mmol), HATU (32 mg, 0.084 mmol), triethylamine (30 ul, 0.212 mmol), 3-methyl-4-(7-methylbenzo[d]oxazol-2-yl)aniline (20 mg, 0.084) and catalytic amount of DMAP in THF (1 mL) was stirred at 65° C. for 48 h. The reaction mixture was diluted with DCM, washed with saturated solution of NaHCO₃ and brine. The organic solution was, dried over Na₂SO₄, decanted and evaporated under reduced pressure. The crude was purified by column chromatography on silica gel using 1:4 EtOAc:Hexane as mobile phase and the obtained product was triturated in hexane, filtered and dried to give N-(3-methyl-4-(7-methylbenzo[d]oxazol-2-yl)phenyl)isoxazole-3-carboxamide (3 mg, 10%). UPLC-MS (Acidic Method, 4 min): rt 2.12 min, m/z 334.1 [M+H]⁺

¹H NMR (400 MHz, METHANOL-d₄) δ ppm 7.32 (d, J=1.6 Hz, 1H), 6.63 (d, J=8.5 Hz, 1H), 6.26-6.32 (m, 2H), 6.02 (d, J=7.9 Hz, 1H), 5.71-5.77 (m, 1H), 5.65-5.69 (m, 1H), 5.40 (d, J=1.6 Hz, 1H), 1.27 (s, 3H), 1.06 (s, 3H).

### Example 9 Preparation of 1-methyl-N-(3-methyl-4-(7-methylbenzo[d]oxazol-2-yl)phenyl)-1H-pyrazole-5-carboxamide

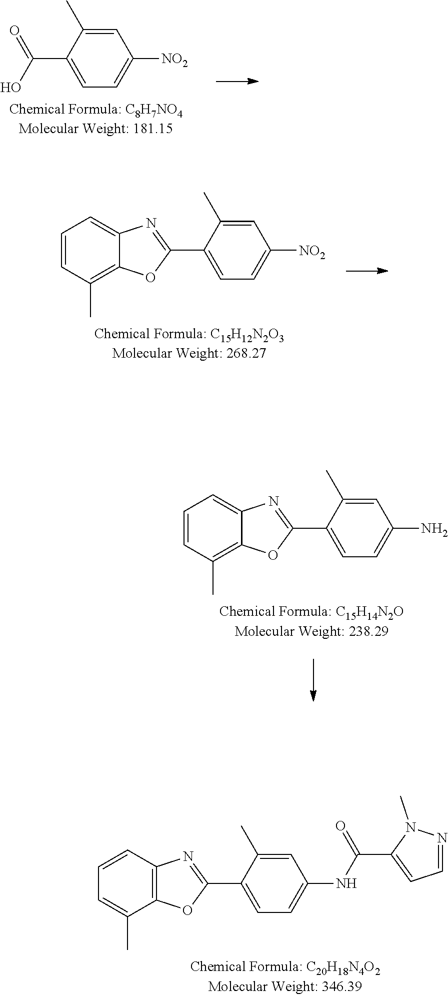 [SMILES: Cc1cc([N+](=O)[O-])ccc1C(=O)O ; Cc1cc([N+](=O)[O-])ccc1-c1nc2cccc(C)c2o1 ; Cc1cc(N)ccc1-c1nc2cccc(C)c2o1 ; Cc1cc(NC(=O)c2ccnn2C)ccc1-c1nc2cccc(C)c2o1]

### 7-methyl-2-(2-methyl-4-nitrophenyl)benzo[d]oxazole

Compound prepared according to general METHOD 4 to give 7-methyl-2-(2-methyl-4-nitrophenyl)benzo[d]oxazole. UPLC-MS (Acidic Method, 4 min): rt 2.33 min, m/z 269.2 [M+H]⁺

### 3-methyl-4-(7-methylbenzo[d]oxazol-2-yl)aniline

Compound prepared according to general METHOD 3 to give 3-methyl-4-(7-methylbenzo [d]oxazol-2-yl)aniline. UPLC-MS (Acidic Method, 2 min): rt 1.17 min, m/z 239.2 [M+H]⁺

### 1-methyl-N-(3-methyl-4-(7-methylbenzo[d]oxazol-2-yl)phenyl)-1H-pyrazole-5-carboxamide

A solution of 1-methyl-1H-pyrazole-5-carboxylic acid (26 mg, 0.21 mmol), HATU (80 mg, 0.21 mmol), triethylamine (74 ul, 0.525 mmol), 3-methyl-4-(7-methylbenzo[d]oxazol-2-yl)aniline (50 mg, 0.21) and catalytic amount of DMAP in THF (1 mL) was stirred at room temperature for 18 h. The reaction mixture was diluted with DCM, washed with saturated solution of NaHCO₃ and brine. The organic solution was, dried over Na₂SO₄, decanted and evaporated under reduced pressure. The crude was purified by column chromatography on silica gel using 1:2 EtOAc:Hexane as mobile phase to give 1-methyl-N-(3-methyl-4-(7-methylbenzo[d]oxazol-2-yl)phenyl)-1H-pyrazole-5-carboxamide (16 mg, 25%). UPLC-MS (Acidic Method, 4 min): rt 2.07 min, m/z 347.1 [M+H]⁺

¹H NMR (400 MHz, DMSO-d₆) δ ppm 10.42 (s, 1H), 8.15-8.21 (m, 1H), 7.81-7.87 (m, 2H), 7.59-7.64 (m, 1H), 7.54-7.58 (m, 1H), 7.27-7.33 (m, 1H), 7.21-7.26 (m, 1H), 7.13 (d, J=2.1 Hz, 1H), 4.11 (s, 3H), 2.77 (s, 3H), 2.53-2.59 (m, 3H)

### Example 10 Preparation of N-(4-(benzo[d]oxazol-2-yl)phenyl)-1-methyl-1H-pyrazole-5-carboxamide

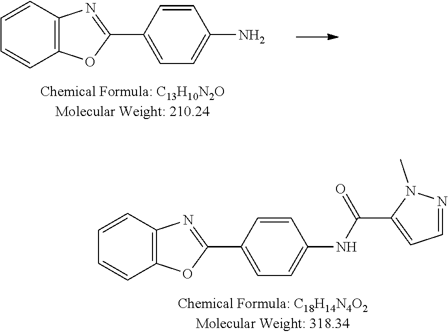 [SMILES: Nc1ccc(-c2nc3ccccc3o2)cc1 ; Cn1nccc1C(=O)Nc1ccc(-c2nc3ccccc3o2)cc1]

A solution of 1-methyl-1H-pyrazole-5-carboxylic acid (34 mg, 0.269 mmol), HBTU (102 mg, 0.269 mmol), triethylamine (93 ul, 0.672 mmol), 4-(benzo[d]oxazol-2-yl)aniline (56 mg, 0.269) and catalytic amount of DMAP in DCM:DMF (3:1 mL) was stirred at room temperature for 24 h. The reaction mixture was diluted with DCM, washed with saturated solution of NaHCO₃ and brine. The organic solution was, dried over Na₂SO₄, decanted and evaporated under reduced pressure. The crude was purified by column chromatography on silica gel using 2:3 EtOAc:Hexane as mobile phase to give N-(4-(benzo[d]oxazol-2-yl)phenyl)-1-methyl-1H-pyrazole-5-carboxamide (14 mg, 16%). UPLC-MS (Acidic Method, min): rt 1.75 min, m/z 319.1 [M+H]⁺

¹H NMR (400 MHz, DMSO-d₆) δ ppm 10.52 (s, 1H), 8.19-8.23 (m, 2H), 7.98-8.03 (m, 2H), 7.76-7.81 (m, 2H), 7.57 (d, J=2.1 Hz, 1H), 7.38-7.45 (m, 2H), 7.13 (d, J=2.1 Hz, 1H), 4.11 (s, 3H).

### Example 11 Preparation of N-(4-(7-chlorobenzo[d]oxazol-2-yl)phenyl)-1-methyl-1H-pyrazole-5-carboxamide

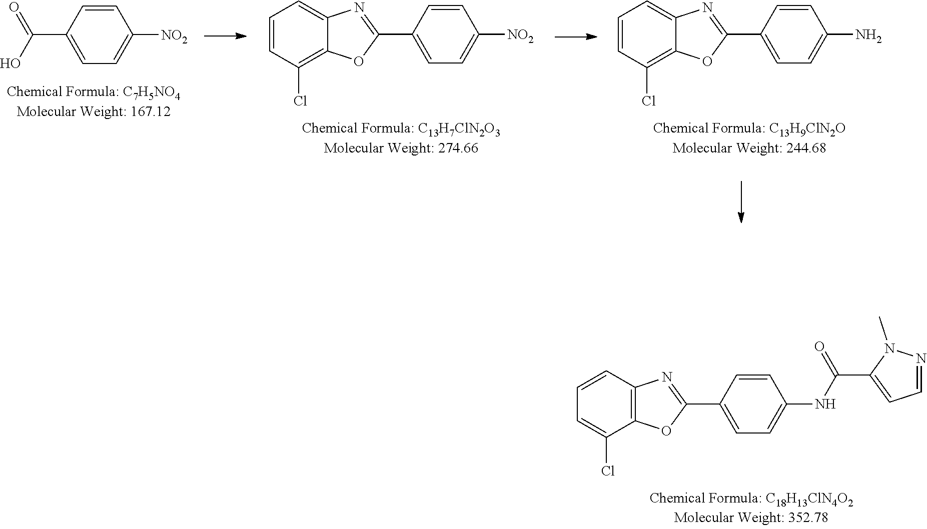 [SMILES: O=C(O)c1ccc([N+](=O)[O-])cc1 ; O=[N+]([O-])c1ccc(-c2nc3cccc(Cl)c3o2)cc1 ; Nc1ccc(-c2nc3cccc(Cl)c3o2)cc1 ; Cn1nccc1C(=O)Nc1ccc(-c2nc3cccc(Cl)c3o2)cc1]

### chloro-2-(4-nitrophenyl)benzo[d]oxazole

To a solution of 4-nitrobenzoic acid (232 mg, 1.39 mmol) in DCM (3 mL) containing 1 drop of DMF was added oxalyl chloride (1.78 uL, 2.09 mmol) drop wise and the suspension was stirred at room temperature for 1.5 hours to form a clear solution. Volatiles were removed under vacuum and to the resulting crude acid chloride, was added a suspension of 2-amino-6-chlorophenol (0.2 g, 1.39 mmol) and triethylamine (491 uL, 3.47 mmol) in DCM (3 mL), drop wise, while vigorously stirring, and the reaction was continued at ambient temperature for 18 hours. The obtained suspension volume was reduced to dryness under vacuum, taken up in dioxane (10 mL) and refluxed for 18 hours after adding methylsulfonic acid (180 uL, 2.78 mmol) to it. The reaction mixture was cooled down to room temperature, diluted with DCM, washed with saturated sodium bicarbonate, brine, and the organic liquor was dried over Na₂SO₄, decanted and evaporated under reduced pressure. The crude was triturated in EtOAc, filtered and dried to give 7-chloro-2-(4-nitrophenyl)benzo[d]oxazole (0.255 g, 67%).

UPLC-MS (Acidic Method, 2 min): rt 1.17 min, m/z 275.1 [M+H]⁺

### 4-(7-chlorobenzo[d]oxazol-2-yl)aniline

To a suspension of 7-chloro-2-(4-nitrophenyl)benzo[d]oxazole (170 mg, 0.62 mmol) in MeOH (4 mL) was added 10% Pd/C in a MeOH slurry and the mixture was stirred at room temperature for 2 hours under a hydrogen atmosphere (balloon). The reaction mixture was filtered through celite and the crude mixture was purified by column chromatography on silica gel using 1:5 EtOAc:Hexane as mobile phase to give to give 4-(7-chlorobenzo[d]oxazol-2-yl)aniline (50 mg, 33%). UPLC-MS (Acidic Method, 2 min): rt 1.04 min, m/z 245.1 [M+H]⁺

### N-(4-(7-chlorobenzo[d]oxazol-2-yl)phenyl)-1-methyl-1H-pyrazole-5-carboxamide

mmol), triethylamine (85 ul, 0.60 mmol), 4-(7-chlorobenzo[d]oxazol-2-yl)aniline (50 mg, 0.0.204) and catalytic amount of DMAP in THF (2 mL) was stirred at room temperature for 18 h. The reaction mixture was diluted with DCM, washed with saturated solution of NaHCO₃ and brine. The organic solution was, dried over Na₂SO₄, decanted and evaporated under reduced pressure. The crude was purified by column chromatography on silica gel using 1:2 EtOAc:Hexane as mobile phase to give N-(4-(7-chlorobenzo[d]oxazol-2-yl)phenyl)-1-methyl-1H-pyrazole-5-carboxamide (8 mg, 11%). UPLC-MS (Acidic Method, 4 min): rt 1.83 min, m/z 353.1 [M+H]⁺

¹H NMR (DMSO-d₆) δ: 10.56 (s, 1H), 8.21-8.25 (m, 2H), 8.02-8.06 (m, 21H), 7.78 (dd, J=7.9, 1.0 Hz, 1H), 7.56-7.60 (m, 1H), 7.54-7.55 (m, 1H), 7.41-7.46 (m, 1H), 7.15 (d, J=2.1 Hz, 1H), 4.09-4.13 (m, 3H).

### Example 12 Preparation of N-(2-(1H-indol-3-yl)ethyl)-2-(4-(1-methyl-1H-pyrazole-5-carboxamido)phenyl)benzo[d]oxazole-7-carboxamide

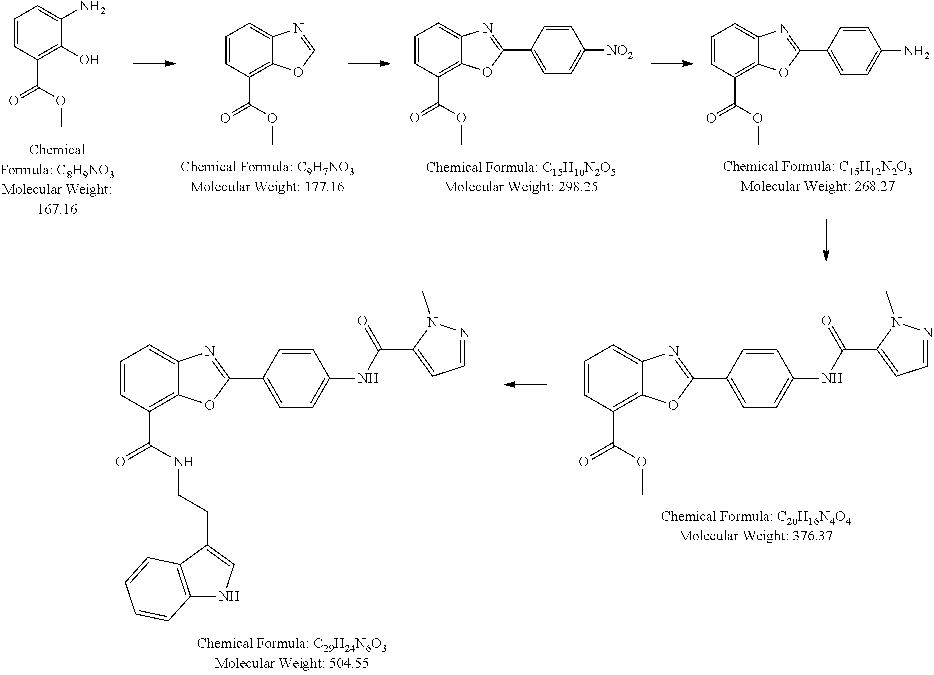 [SMILES: COC(=O)c1cccc(N)c1O ; COC(=O)c1cccc2ncoc12 ; COC(=O)c1cccc2nc(-c3ccc([N+](=O)[O-])cc3)oc12 ; COC(=O)c1cccc2nc(-c3ccc(N)cc3)oc12 ; Cn1nccc1C(=O)Nc1ccc(-c2nc3cccc(C(=O)NCCc4c[nH]c5ccccc45)c3o2)cc1 ; COC(=O)c1cccc2nc(-c3ccc(NC(=O)c4ccnn4C)cc3)oc12]

### Methyl benzo[d]oxazole-7-carboxylate

A solution of methyl 3-amino-2-hydroxybenzoate (1 g, 5.99 mmol) and p-toluenesulfonic acid (0.1 g, 0.581 mmol) in trimethylorthoformate (50 mL) was refluxed for 1 hour. Volatiles were evaporated under reduced pressure. The crude was purified by column chromatography on silica gel using 20:1 DCM:EtOAc as eluent to give methyl benzo[d]oxazole-7-carboxylate (0.99 g, 93%).

UPLC-MS (Acidic Method, 2 min): rt 0.79 min, m/z 178.1 [M+H]⁺

### Methyl 2-(4-nitrophenyl)benzo[d]oxazole-7-carboxylate

A solution of methyl benzo[d]oxazole-7-carboxylate (0.5 g, 2.82 mmol), 1-iodo-4-nitrobenzene (0.88 g, 3.53 mmol), lithium tert-butoxide (0.45 g, 5.64 mmol), tetrakis(triphenyl phosphine)palladium(0) (0.16 g, 0.141 mmol) in dioxane under a nitrogen atmosphere was stirred at room temperature for 45 minutes. The resulting thick suspension was diluted with EtOAc, washed with water, organics washed with brine, dried over Na₂SO₄, decanted and solvent removed under vacuum. The crude was purified by column chromatography on silica gel using 20:1 to 5:1 Hexane:EtOAc as eluent to give methyl 2-(4-nitrophenyl)benzo[d]oxazole-7-carboxylate (0.4 g, 48%).

UPLC-MS (Acidic Method, 2 min): rt 1.11 min, m/z 299.0 [M+H]⁺

### Methyl 2-(4-aminophenyl)benzo[d]oxazole-7-carboxylate

To a suspension of methyl 2-(4-nitrophenyl)benzo[d]oxazole-7-carboxylate (20 mg, 0.067 mmol) in EtOH (1 mL) was added 10% Pd/C and the mixture was stirred at room temperature for 72 hours under a hydrogen atmosphere (balloon). The reaction mixture was filtered through celite and the filtrate volume was reduced to dryness under vacuum to give methyl 2-(4-aminophenyl)benzo[d]oxazole-7-carboxylate (18 mg, 100%).

UPLC-MS (Acidic Method, 2 min): rt 1.02 min, m/z 269.1 [M+H]⁺

### Methyl 2-(4-(1-methyl-1H-pyrazole-5-carboxamido)phenyl)benzo[d]oxazole-7-carboxylate

A solution of 1-methyl-1H-pyrazole-5-carboxylic acid (17 mg, 0.134 mmol), HATU (51 mg, 0.134 mmol), triethylamine (41 mg, 0.402 mmol) and catalytic amount of DMAP in THF (1 mL) was stirred at room temperature for 10 minutes. Methyl 2-(4-aminophenyl)benzo[d]oxazole-7-carboxylate (18 mg, 0.067 mmol) was added and the resulting mixture was stirred at 75° C. for 72 h. The reaction mixture was diluted with DCM, washed with saturated solution of NaHCO₃ and brine. The organic solution was, dried over Na₂SO₄, decanted and evaporated under reduced pressure. The crude was purified by column chromatography on silica gel using 1:1 EtOAc:Hexane as mobile phase. The resulting product was triturated in diethyl ether, filtered and dried to give methyl 2-(4-(1-methyl-1H-pyrazole-5-carboxamido)phenyl)benzo[d]oxazole-7-carboxylate (2 mg, 8%). UPLC-MS (Acidic Method, 4 min): rt 1.77 min, m/z 377.1 [M+H]⁺

¹H NMR (400 MHz, CDCl₃-d) 6 ppm 8.36 (d, J=8.7 Hz, 2H), 7.95-8.03 (m, 2H), 7.83 (br d, J=8.7 Hz, 3H), 7.56 (d, J=1.5 Hz, 1H), 7.45 (s, 1H), 6.72 (s, 1H), 4.24-4.29 (m, 3H), 4.08 (s, 3H)

### N-(2-(1H-indol-3-yl)ethyl)-2-(4-(1-methyl-1H-pyrazole-5-carboxamido)phenyl)benzo[d]oxazole-7-carboxamide

In a sealed vial, methyl 2-(4-(1-methyl-1H-pyrazole-5-carboxamido)phenyl)benzo[d]oxazole-7-carboxylate (100 mg, 0.26 mmol) and tryptamine (208 mg, 1.3 mmol) were stirred in THF at 70° C. for 18 hours. The reaction mixture was allowed to cool to room temperature, diluted with DCM and washed with 1M HCl, water, brine, dried over Na₂SO₄, decanted and columned using 1:1 and 2:3 Hexane:EtOAc; the obtained solid was triturated in Hexane:EtOAc, filtered and dried to give N-(2-(1H-indol-3-yl)ethyl)-2-(4-(1-methyl-1H-pyrazole-5-carboxamido)phenyl)benzo[d]oxazole-7-carboxamide (15 mg, 11%). UPLC-MS (Acidic Method, 4 min): rt 1.71 min, m/z 505.2 [M+H]⁺

¹H NMR (DMSO-d₆) δ: 10.86 (br s, 1H), 10.56 (s, 1H), 8.51 (br t, J=5.5 Hz, 1H), 8.17 (d, J=8.8 Hz, 2H), 8.01 (d, J=8.8 Hz, 2H), 7.89-7.98 (m, 1H), 7.78 (d, J=7.8 Hz, 1H), 7.66 (d, J=7.8 Hz, 1H), 7.58 (d, J=2.0 Hz, 1H), 7.49 (t, J=7.8 Hz, 1H), 7.34-7.41 (m, 1H), 7.23-7.30 (m, 1H), 7.12-7.18 (m, 1H), 7.05-7.12 (m, 1H), 6.93-7.04 (m, 1H), 4.13 (s, 3H), 3.66-3.79 (m, 2H), 3.08 (br t, J=7.3 Hz, 2H).

### Example 13 Preparation of N-(4-(7-(hydroxymethyl)benzo[d]oxazol-2-yl)phenyl)-1-methyl-1H-pyrazole-5-carboxamide

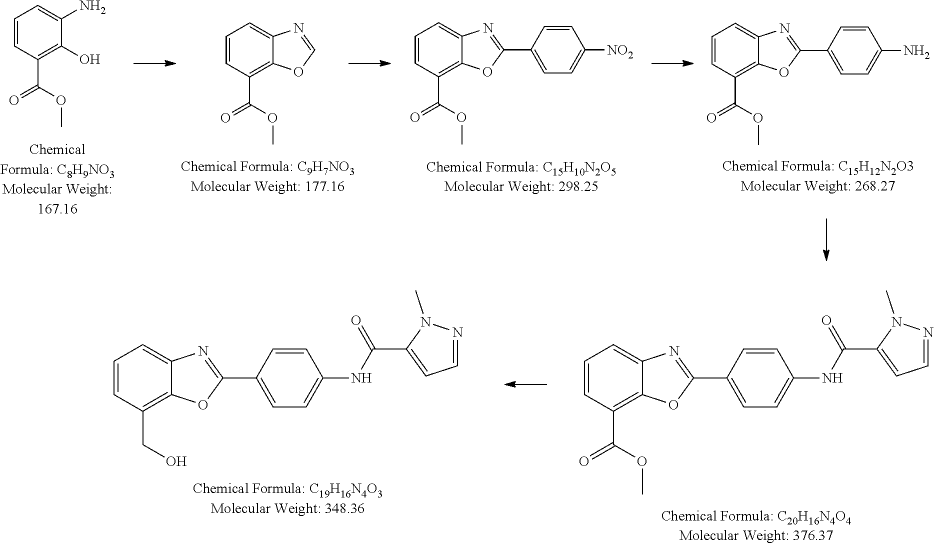 [SMILES: COC(=O)c1cccc(N)c1O ; COC(=O)c1cccc2ncoc12 ; COC(=O)c1cccc2nc(-c3ccc([N+](=O)[O-])cc3)oc12 ; COC(=O)c1cccc2nc(-c3ccc(N)cc3)oc12 ; Cn1nccc1C(=O)Nc1ccc(-c2nc3cccc(CO)c3o2)cc1 ; COC(=O)c1cccc2nc(-c3ccc(NC(=O)c4ccnn4C)cc3)oc12]

### Methyl benzo[d]oxazole-7-carboxylate

A solution of methyl 3-amino-2-hydroxybenzoate (1 g, 5.99 mmol) and p-toluenesulfonic acid (0.1 g, 0.581 mmol) in trimethylorthoformate (50 mL) was refluxed for 1 hour. Volatiles were evaporated under reduced pressure. The crude was purified by column chromatography on silica gel using 20:1 DCM:EtOAc as eluent to give methyl benzo[d]oxazole-7-carboxylate (0.99 g, 93%).

UPLC-MS (Acidic Method, 2 min): rt 0.79 min, m/z 178.1 [M+H]⁺

### Methyl 2-(4-nitrophenyl)benzo[d]oxazole-7-carboxylate

A solution of methyl benzo[d]oxazole-7-carboxylate (0.5 g, 2.82 mmol), 1-iodo-4-nitrobenzene (0.88 g, 3.53 mmol), lithium tert-butoxide (0.45 g, 5.64 mmol), tetrakis(triphenyl phosphine)palladium(0) (0.16 g, 0.141 mmol) in dioxane under a nitrogen atmosphere was stirred at room temperature for 45 minutes. The resulting thick suspension was diluted with EtOAc, washed with water, organics washed with brine, dried over Na₂SO₄, decanted and solvent removed under vacuum. The crude was purified by column chromatography on silica gel using 20:1 to 5:1 Hexane:EtOAc as eluent to give methyl 2-(4-nitrophenyl)benzo[d]oxazole-7-carboxylate (0.4 g, 48%). UPLC-MS (Acidic Method, 2 min): rt 1.11 min, m/z 299.0 [M+H]⁺

### Methyl 2-(4-aminophenyl)benzo[d]oxazole-7-carboxylate

To a suspension of methyl 2-(4-nitrophenyl)benzo[d]oxazole-7-carboxylate (20 mg, 0.067 mmol) in EtOH (1 mL) was added 10% Pd/C and the mixture was stirred at room temperature for 72 hours under a hydrogen atmosphere (balloon). The reaction mixture was filtered through celite and the filtrate volume was reduced to dryness under vacuum to give methyl 2-(4-aminophenyl)benzo[d]oxazole-7-carboxylate (18 mg, 100%).

UPLC-MS (Acidic Method, 2 min): rt 1.02 min, m/z 269.1 [M+H]⁺

### Methyl 2-(4-(1-methyl-1H-pyrazole-5-carboxamido)phenyl)benzo[d]oxazole-7-carboxylate

A solution of 1-methyl-1H-pyrazole-5-carboxylic acid (17 mg, 0.134 mmol), HATU (51 mg, 0.134 mmol), triethylamine (41 mg, 0.402 mmol) and catalytic amount of DMAP in THF (1 mL) was stirred at room temperature for 10 minutes. Methyl 2-(4-aminophenyl)benzo[d]oxazole-7-carboxylate (18 mg, 0.067 mmol) was added and the resulting mixture was stirred at 75° C. for 72 h. The reaction mixture was diluted with DCM, washed with saturated solution of NaHCO₃ and brine. The organic solution was, dried over Na₂SO₄, decanted and evaporated under reduced pressure. The crude was purified by column chromatography on silica gel using 1:1 EtOAc:Hexane as mobile phase. The resulting product was triturated in diethyl ether, filtered and dried to give methyl 2-(4-(1-methyl-1H-pyrazole-5-carboxamido)phenyl)benzo[d]oxazole-7-carboxylate (2 mg, 8%). UPLC-MS (Acidic Method, 4 min): rt 1.77 min, m/z 377.1 [M+H]⁺

¹H NMR (400 MHz, CDCl₃-d) δ ppm 8.36 (d, J=8.7 Hz, 2H), 7.95-8.03 (m, 2H), 7.83 (br d, J=8.7 Hz, 3H), 7.56 (d, J=1.5 Hz, 1H), 7.45 (s, 1H), 6.72 (s, 1H), 4.24-4.29 (m, 3H), 4.08 (s, 3H)

### N-(4-(7-(hydroxymethyl)benzo[d]oxazol-2-yl)phenyl)-1-methyl-1H-pyrazole-5-carboxamide

LiBH₄ (18 mg, 0.798 mmol) was added to a solution of methyl 2-(4-(1-methyl-1H-pyrazole-5-carboxamido)phenyl)benzo[d]oxazole-7-carboxylate (100 mg, 0.266 mmol) in 2-methyl-THF under a nitrogen atmosphere and the mixture was stirred at 40° C. for 30 minutes. Once the solution reached room temperature it was quenched with 3 mL of water and 50 mg of solid NaOH and EtOAc was added and the bi-phasic mixture stirred vigorously for 10 minutes. The organic layer was washed with brine, dried over Na₂SO₄, decanted and columned over silica using 1:1 and 3:2 EtOAc:Hexane. The obtained solid was triturated in EtOAc, filtered and dried to give N-(4-(7-(hydroxymethyl)benzo[d]oxazol-2-yl)phenyl)-1-methyl-1H-pyrazole-5-carboxamide (15 mg, 16%). UPLC-MS (Acidic Method, 4 min): rt 1.32 min, m/z 349.1 [M+H]⁺

¹H NMR (DMSO-d₆) δ: 10.53 (s, 1H), 8.21-8.26 (m, 2H), 7.99-8.04 (m, 2H), 7.68 (dd, J=7.6, 1.4 Hz, 1H), 7.57 (d, J=2.0 Hz, 1H), 7.36-7.45 (m, 2H), 7.14 (d, J=2.1 Hz, 1H), 5.45 (t, J=5.8 Hz, 1H), 4.87 (d, J=5.8 Hz, 2H), 4.12 (s, 3H).

### Example 14 Preparation of 2-(4-(1-methyl-1H-pyrazole-5-carboxamido)phenyl) benzo [d]oxazole-7-carboxamide

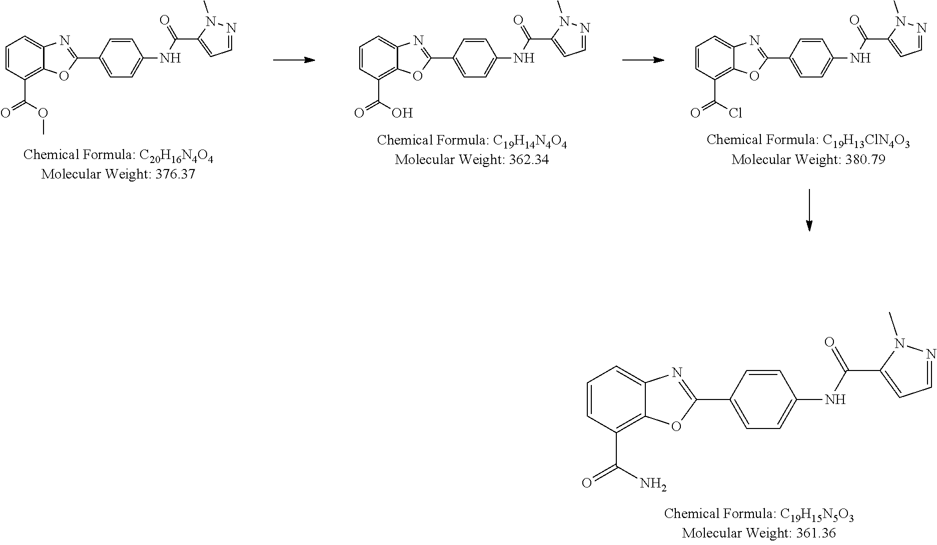 [SMILES: COC(=O)c1cccc2nc(-c3ccc(NC(=O)c4ccnn4C)cc3)oc12 ; Cn1nccc1C(=O)Nc1ccc(-c2nc3cccc(C(=O)O)c3o2)cc1 ; Cn1nccc1C(=O)Nc1ccc(-c2nc3cccc(C(=O)Cl)c3o2)cc1 ; Cn1nccc1C(=O)Nc1ccc(-c2nc3cccc(C(N)=O)c3o2)cc1]

### Methyl 2-(4-(1-methyl-1H-pyrazole-5-carboxamido)phenyl)benzo[d]oxazole-7-carboxylate as described for Example 13

### 2-(4-(1-methyl-1H-pyrazole-5-carboxamido)phenyl)benzo[d]oxazole-7-carboxylic acid

A solution of LiOH (112 mg, 2.66 mmol) in water (5 mL) was added to a solution of methyl 2-(4-(1-methyl-1H-pyrazole-5-carboxamido)phenyl)benzo[d]oxazole-7-carboxylate (200 mg, 0.532) in THF (5 mL) and the resulting mixture was stirred at RT for 1 hour. THF was removed under vacuum and conc. HCl was added to acidic pH. The formed suspension was filtered and the obtained solid was dried to give 2-(4-(1-methyl-1H-pyrazole-5-carboxamido)phenyl) benzo[d]oxazole-7-carboxylic acid (155 mg, 78%).

UPLC-MS (Acidic Method, 2 min): rt 0.90 min, m/z 363.2 [M+H]⁺

### 2-(4-(1-methyl-1H-pyrazole-5-carboxamido)phenyl)benzo[d]oxazole-7-carbonyl chloride

Oxalyl chloride (55 ul, 0.642 mmol) was added drop wise to a solution of 2-(4-(1-methyl-1H-pyrazole-5-carboxamido)phenyl)benzo[d]oxazole-7-carboxylic acid (155 mg, 0.428 mmol) in DCM (5 mL) and DMF (1 drop) and the mixture was stirred at room temperature for 1 hour. Volatiles were removed under vacuum to give 2-(4-(1-methyl-1H-pyrazole-5-carboxamido)phenyl) benzo[d]oxazole-7-carbonyl chloride (170 mg, 100%).

¹H NMR (DMSO-d₆) δ: 12.47-12.86 (m, 1H), 10.62 (s, 1H), 8.18-8.24 (m, 2H), 8.02-8.09 (m, 3H), 7.88-7.94 (m, 1H), 7.55-7.59 (m, 1H), 7.47-7.55 (m, 1H), 7.19 (d, J=2.1 Hz, 1H), 4.12 (s, 3H)

### 2-(4-(1-methyl-1H-pyrazole-5-carboxamido)phenyl)benzo[d]oxazole-7-carboxamide

2-(4-(1-methyl-1H-pyrazole-5-carboxamido)phenyl)benzo[d]oxazole-7-carbonyl chloride (150 mg, 0.395 mmol) was added portion wise to a 0.5M solution of ammonia in dioxane (8 mL) and the resulting mixture was stirred at RT for 18 hours. Volatiles were removed under vacuum and the crude material was purified over silica using 3% and 5% MeOH in DCM to give 2-(4-(1-methyl-1H-pyrazole-5-carboxamido)phenyl)benzo[d]oxazole-7-carboxamide (11 mg, 8%).

UPLC-MS (Acidic Method, 4 min): rt 1.32 min, m/z 362.1 [M+H]⁺

¹H NMR (DMSO-d₆) δ: 10.55 (s, 1H), 8.30-8.34 (m, 2H), 8.01-8.05 (m, 2H), 7.92-7.98 (m, 1H), 7.84-7.92 (m, 2H), 7.77-7.84 (m, 1H), 7.58 (d, J=2.1 Hz, 1H), 7.49 (t, J=7.8 Hz, 1H), 7.14 (d, J=2.0 Hz, 1H), 4.11-4.15 (m, 3H)

### Example 15 Preparation of N-Ethyl-2-(4-(1-methyl-1H-pyrazole-5-carboxamido) phenyl) benzo[d]oxazole-7-carboxamide

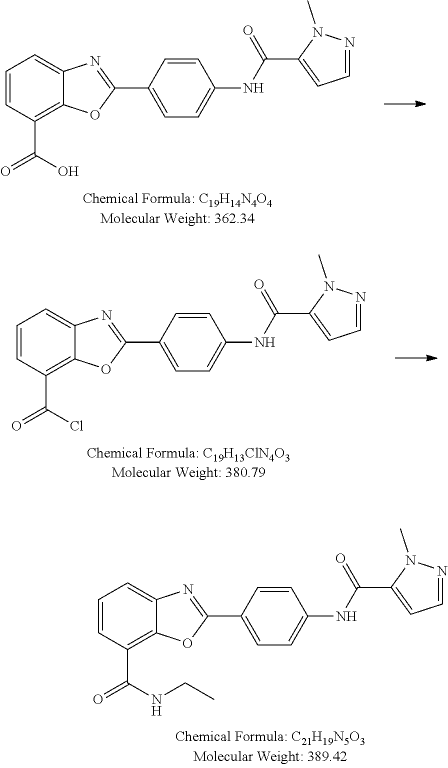 [SMILES: Cn1nccc1C(=O)Nc1ccc(-c2nc3cccc(C(=O)O)c3o2)cc1 ; Cn1nccc1C(=O)Nc1ccc(-c2nc3cccc(C(=O)Cl)c3o2)cc1 ; CCNC(=O)c1cccc2nc(-c3ccc(NC(=O)c4ccnn4C)cc3)oc12]

### 2-(4-(1-methyl-1H-pyrazole-5-carboxamido)phenyl)benzo[d]oxazole-7-carbonyl chloride as described for Example 14

### N-ethyl-2-(4-(1-ethyl-1H-pyrazole-5-carboxamido)phenyl)benzo[d]oxazole-7-carboxamide

2-(4-(1-methyl-1H-pyrazole-5-carboxamido)phenyl)benzo[d]oxazole-7-carbonyl chloride (100 mg, 0.262 mmol) was stirred in 2M ethylamine in THF (1.3 mL, 2.6 mmol) at room temperature for 18 hours. The reaction mixture was filtered and the obtained solid was purified over silica using 3% and 5% MeOH in DCM to give N-ethyl-2-(4-(1-methyl-1H-pyrazole-5-carboxamido)phenyl)benzo[d]oxazole-7-carboxamide (2 mg, 3%). UPLC-MS (Acidic Method, 4 min): rt 1.52 min, m/z 390.2 [M+H]⁺

¹H NMR (DMSO-d₆) δ: 10.56 (s, 1H), 8.46 (s, 1H), 8.26-8.30 (m, 2H), 8.01-8.06 (m, 2H), 7.93 (dd, J=7.9, 1.1 Hz, 1H), 7.75 (dd, J=7.7, 1.1 Hz, 1H), 7.58 (d, J=2.1 Hz, 1H), 7.48 (t, J=7.8 Hz, 1H), 7.14 (d, J=2.0 Hz, 1H), 4.12 (s, 31H), 3.43 (dd, J=7.2, 5.6 Hz, 21H), 1.21-1.28 (m, 31H).

### Example 16 Preparation of N-(4-(7-Cyanobenzo[d]oxazol-2-yl)phenyl)-1-methyl-1H-pyrazole-5-carboxamide

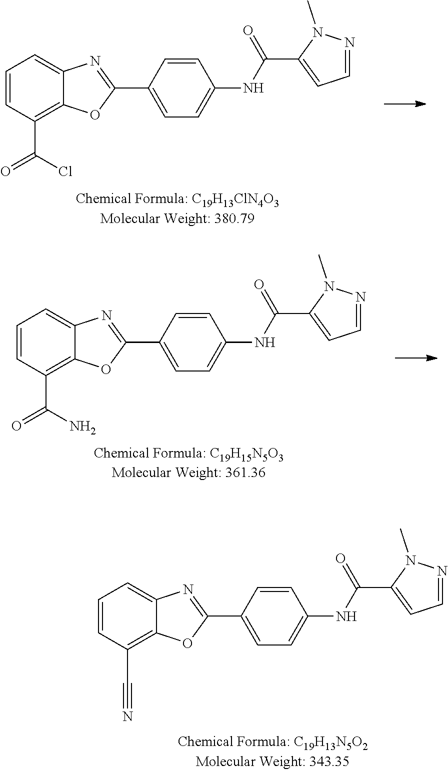 [SMILES: Cn1nccc1C(=O)Nc1ccc(-c2nc3cccc(C(=O)Cl)c3o2)cc1 ; Cn1nccc1C(=O)Nc1ccc(-c2nc3cccc(C(N)=O)c3o2)cc1 ; Cn1nccc1C(=O)Nc1ccc(-c2nc3cccc(C#N)c3o2)cc1]

### 2-(4-(1-Methyl-1H-pyrazole-5-carboxamido)phenyl)benzo[d]oxazole-7-carboxamide as described for Example 14

### N-(4-(7-Cyanobenzo[d]oxazol-2-yl)phenyl)-1-methyl-1H-pyrazole-5-carboxamide

Triethylamine (45 mg, 0.441 mmol) and trifluoroacetic anhydride (31 mg, 0.147 mmol) were added step wise to a suspension of 2-(4-(1-methyl-1H-pyrazole-5-carboxamido)phenyl) benzo[d]oxazole-7-carboxamide (32 mg, 0.123 mmol) in DCM (3 mL) and the mixture was stirred at room temperature for 18 hours. The reaction mixture was then diluted with DCM, washed with water, brine and the organic liquor was dried over Na₂SO₄ decanted and purified by column over silica gel using 1:1 Hexane:EtOAc to give N-(4-(7-cyanobenzo[d]oxazol-2-yl)phenyl)-1-methyl-1H-pyrazole-5-carboxamide (2 mg, 7%).

UPLC-MS (Acidic Method, 4 min): rt 1.76 min, m/z 344.2 [M+H]⁺

¹H NMR (CDCl₃-d) δ: 8.31 (d, J=8.7 Hz, 21H), 7.99 (br s, 1H), 7.90 (d, J=8.8 Hz, 21H), 7.61-7.68 (m, 1H), 7.52-7.58 (m, 1H), 7.41-7.50 (m, 1H), 6.80-6.87 (m, 1H), 4.19-4.29 (m, 3H)

### Example 17 Preparation of 1-methyl-N-(4-(4-methylbenzo[d]oxazol-2-yl)phenyl)-1H-pyrazole-5-carboxamide

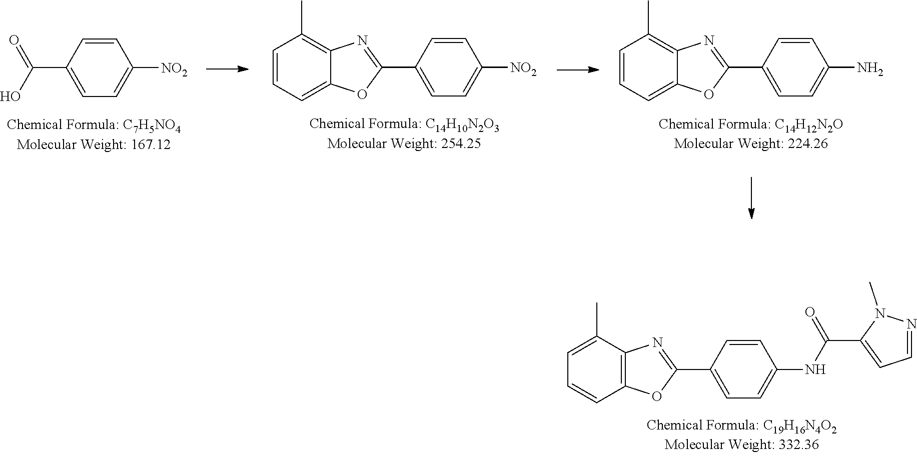 [SMILES: O=C(O)c1ccc([N+](=O)[O-])cc1 ; Cc1cccc2oc(-c3ccc([N+](=O)[O-])cc3)nc12 ; Cc1cccc2oc(-c3ccc(N)cc3)nc12 ; Cc1cccc2oc(-c3ccc(NC(=O)c4ccnn4C)cc3)nc12]

### 4-Methyl-2-(4-nitrophenyl)benzo[d]oxazole

To a solution of 4-nitrobenzoic acid (1.36 g, 8.13 mmol) in DCM (15 mL) containing 5 drops of DMF was added oxalyl chloride (1.04 mL, 12.2 mmol) drop wise and the suspension was stirred at room temperature for 1.5 hours to form a clear solution. Volatiles were removed under vacuum and to the resulting crude acid chloride, was added a suspension of 2-amino-m-cresol (1 g, 8.13 mmol) and triethylamine (2.9 mL, 20.3 mmol) in DCM (15 mL), drop wise, while vigorously stirring, and the reaction was continued at ambient temperature for 18 hours. The obtained suspension volume was reduced to dryness under vacuum, taken up in dioxane (20 mL) and refluxed for 3 hours after adding methylsulfonic acid (1.6 mL, 24.4 mmol) to it. The reaction mixture was filtered once cold and the crude solid was washed with hexane and dried to give 4-methyl-2-(4-nitrophenyl)benzo[d]oxazole (1.18 g, 57%). UPLC-MS (Acidic Method, 2 min): rt 1.30 min, m/z 255.2 [M+H]⁺

### 4-(4-Methylbenzo[d]oxazol-2-yl)aniline

To a suspension of 4-methyl-2-(4-nitrophenyl)benzo[d]oxazole (1.18 g, 4.64 mmol) in EtOH (40 mL) was added 10% Pd/C (0.2 g) in an EtOH slurry and the mixture was stirred at room temperature for 18 hours under a hydrogen atmosphere (balloon). The reaction mixture was filtered through celite and the filtrate was vac. down to dryness to give 4-(4-methylbenzo[d]oxazol-2-yl)aniline (1.04 g, 100%).

UPLC-MS (Acidic Method, 2 min): rt 1.12 min, m/z 225.2 [M+H]⁺.

### 1-Methyl-N-(4-(4-methylbenzo[d]oxazol-2-yl)phenyl)-1H-pyrazole-5-carboxamide

A solution of 1-methyl-1H-pyrazole-5-carboxylic acid (113 mg, 0.893 mmol), HATU (339 mg, 0.893 mmol), triethylamine (380 ul, 2.68 mmol), 4-(4-methylbenzo[d]oxazol-2-yl)aniline (200 mg, 0.893) and catalytic amount of DMAP in THF (5 mL) was stirred at 70° C. for 18 h. The reaction mixture was diluted with DCM, washed with saturated solution of NaHCO₃ and brine. The organic solution was, dried over Na₂SO₄, decanted and evaporated under reduced pressure. The crude was purified by column chromatography on silica gel using 1:1 Hexane:EtOAc as mobile phase. The obtained solid was triturated in EtOAc, filtered and dried to give 1-methyl-N-(4-(4-methylbenzo[d]oxazol-2-yl)phenyl)-1H-pyrazole-5-carboxamide (12 mg, 4%).

UPLC-MS (Acidic Method, 4 min): rt 1.98 min, m/z 333.2 [M+H]⁺

¹H NMR (DMSO-d₆) δ: 10.52 (s, 1H), 8.20-8.24 (m, 2H), 7.99-8.04 (m, 2H), 7.55-7.60 (m, 2H), 7.28-7.34 (m, 1H), 7.20-7.25 (m, 1H), 7.14 (d, J=2.0 Hz, 1H), 4.12 (s, 3H), 2.60 (s, 3H).

### Example 18 Preparation of N-(4-(7-(2-hydroxypropan-2-yl)benzo[d]oxazol-2-yl) phenyl)-1-methyl-1H-pyrazole-5-carboxamide

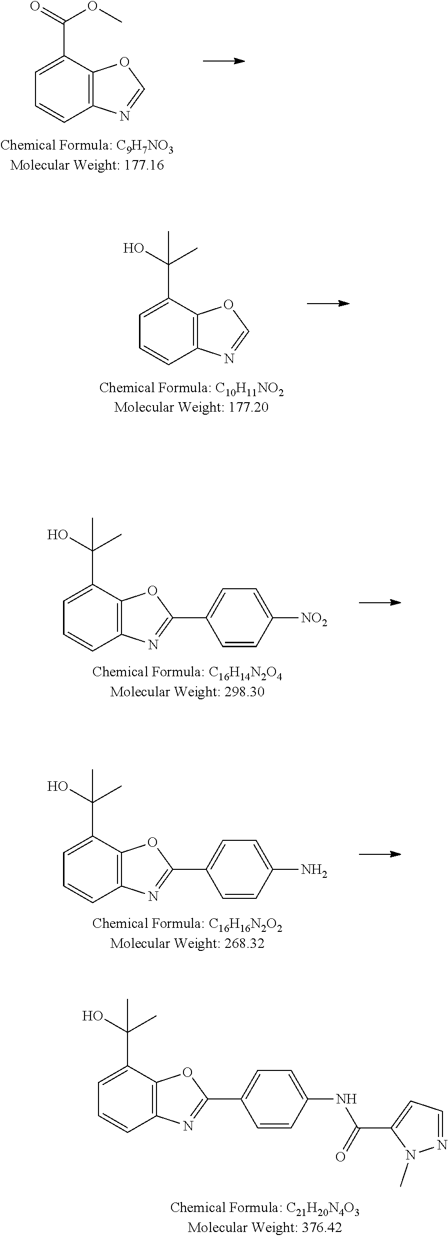 [SMILES: COC(=O)c1cccc2ncoc12 ; CC(C)(O)c1cccc2ncoc12 ; CC(C)(O)c1cccc2nc(-c3ccc([N+](=O)[O-])cc3)oc12 ; CC(C)(O)c1cccc2nc(-c3ccc(N)cc3)oc12 ; Cn1nccc1C(=O)Nc1ccc(-c2nc3cccc(C(C)(C)O)c3o2)cc1]

### 2-(benzo[d]oxazol-7-yl)propan-2-ol

A solution of methyl benzo[d]oxazole-7-carboxylate (300 mg, 1.7 mmol) in THF (5 mL) was cooled down to 0° C. and methyl magnesium bromide 3.2M in 2-Me-THF (3.0 equiv., 5.1 mmol) was added dropwise. The mixture was allowed to warm-up to room temperature and quenched with saturated solution of NH₄Cl. The resulting mixture was extracted 3 times with EtOAc and the organic solutions were washed with brine, dried with Na₂SO₄ and concentrated to dryness. The crude was purified by column chromatography on silica gel using DCM:EtOAc (95:5) as mobile phase to give 2-(benzo[d]oxazol-7-yl)propan-2-ol (110 mg, 37%).

UPLC-MS (Acidic Method, 2 min): rt 0.78 min, m/z 178.0 [M+H]⁺

### 2-(2-(4-nitrophenyl)benzo[d]oxazol-7-yl)propan-2-ol

A solution of 2-(benzo[d]oxazol-7-yl)propan-2-ol (110 mg, 0.62 mmol), iodo 4-nitrobenzene (1.2 equiv., 0.75 mmol), lithium tert-butoxide (2.0 equiv., 1.2 mmol) and Pd(PPh₃)₄(0.05 equiv., 0.03 mmol) in dioxane (6 mL) was stirred under reflux for 3 hours. The reaction mixture was diluted with water and extracted twice with EtOAc. The organic solution was washed with brine solution, dried over Na₂SO₄ and evaporated under reduced pressure. The crude was purified by column chromatography on silica gel using ethyl acetate in hexane as mobile phase to give 2-(2-(4-nitrophenyl)benzo[d]oxazol-7-yl)propan-2-ol (137 mg, 74%) as a yellow oil (83% purity).

UPLC-MS (Basic Method, 2 min): rt 1.12 min, m/z 299.0 [M+H]⁺

¹H NMR (DMSO-d₆) δ: 8.72 (s, 1H), 8.46 (s, 4H), 7.72-7.77 (m, 1H), 7.60 (s, 21H), 7.51-7.56 (m, 1H), 7.43 (t, J=7.8 Hz, 1H), 7.32-7.38 (m, 1H), 5.46 (s, 1H), 5.36 (s, 1H), 1.69 (s, 6H), 1.59 (s, 6H), 0.82-0.89 (m, 9H)

### 2-(2-(4-aminophenyl)benzo[d]oxazol-7-yl)propan-2-ol

To a solution of 2-(2-(4-nitrophenyl)benzo[d]oxazol-7-yl)propan-2-ol (137 mg, 0.46 mmol) in ethanol (5 mL) under N₂ was added Pd/C (50% w/w). The resulting mixture was stirred at room temperature under H₂ for 18 h. The reaction mixture was filtrated on celite pad and rinsed with ethanol. The filtrate was evaporated under reduced pressure to give 2-(2-(4-aminophenyl)benzo[d]oxazol-7-yl)propan-2-ol (40 mg, 32%) as a yellow oil (87% purity). The crude compound was taken to the next step without further purification.

UPLC-MS (Basic Method, 2 min): rt 0.91 min, m/z 269.1 [M+H]⁺

¹H NMR (DMSO-d₆) δ: 7.83 (s, 21H), 7.48-7.52 (m, 1H), 7.40-7.44 (m, 1H), 7.23-7.29 (m, 1H), 6.68-6.73 (m, 21H), 1.65 (s, 6H)

### N-(4-(7-(2-hydroxypropan-2-yl)benzo[d]oxazol-2-yl)phenyl)-1-methyl-1H-pyrazole-5-carboxamide-618-67

A solution of 1-methyl-1H-pyrazole-5-carboxylic acid (19 mg, 0.15 mmol), HATU (57 mg, 1.0 equiv.) and triethylamine (62 μL, 3.0 equiv.) in THF (1.5 mL) was stirred at room temperature for 15 mins, then 2-(2-(4-aminophenyl)benzo[d]oxazol-7-yl)propan-2-ol (40 mg, 1.0 equiv.) and catalytic amount of DMAP were added and the resulting mixture was stirred at 60° C. for 18 h. Small conversion observed by LCMS. 1-methyl-1H-pyrazole-5-carboxylic acid (19 mg, 1.0 equiv.) and HATU (57 mg, 1.0 equiv.) were added and the mixture was stirred at 60° C. for another 4 hours to completion. The reaction mixture was diluted with DCM, washed with saturated solution of NaHCO₃ and brine. The organic solution was, dried over Na₂SO₄, decanted and evaporated under reduced pressure. The crude was purified by column chromatography on silica gel using DCM:MeOH (95:5) as mobile phase to give N-(4-(7-(2-hydroxypropan-2-yl)benzo[d]oxazol-2-yl)phenyl)-1-methyl-1H-pyrazole-5-carboxamide (6 mg, 11%).

UPLC-MS (Basic Method, 2 min): rt 1.03 min, m/z 377.1 [M+H]⁺

¹H NMR (CHLOROFORM-d) δ: 8.27 (d, J=8.4 Hz, 21H), 7.81 (br d, J=8.4 Hz, 21H), 7.68 (d, J=7.8 Hz, 1H), 7.47-7.56 (m, 21H), 7.32-7.39 (m, 1H), 6.75 (s, 1H), 4.25 (s, 31H), 1.84 (s, 6H)

### Example 19 Preparation of N-(4-(7-chloro-1H-benzo[d]imidazol-2-yl)phenyl)-1-methyl-1H-pyrazole-5-carboxamide

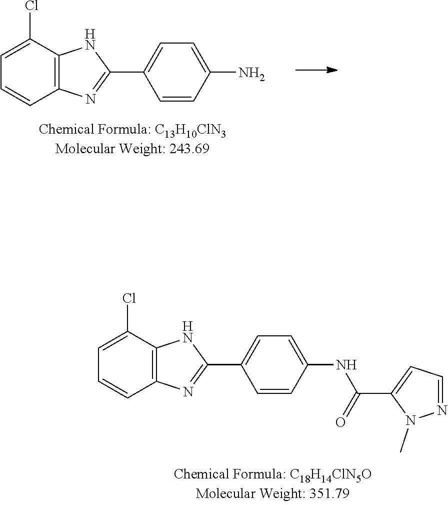 [SMILES: Nc1ccc(-c2nc3cccc(Cl)c3[nH]2)cc1 ; Cn1nccc1C(=O)Nc1ccc(-c2nc3cccc(Cl)c3[nH]2)cc1]

A solution of 1-methyl-1H-pyrazole-5-carboxylic acid (52 mg, 0.41 mmol), HATU (156 mg, 1.0 equiv.) and triethylamine (172 μL, 3.0 equiv.) in THF (4 mL) was stirred at room temperature for 15 mins, then 4-(7-chloro-1H-benzo[d]imidazol-2-yl)aniline (100 mg, 1.0 equiv.) and catalytic amount of DMAP were added and the resulting mixture was stirred at 60° C. for 18 h. Reaction not complete by LCMS. 1-methyl-1H-pyrazole-5-carboxylic acid (52 mg, 1.0 equiv.) and HATU (156 mg, 1.0 equiv.) were added and the mixture was stirred at 60° C. for another 4 hours to completion. The reaction mixture was diluted with DCM, washed with saturated solution of NaHCO₃ and brine. The organic solution was, dried over Na₂SO₄, decanted and evaporated under reduced pressure. The crude was purified by column chromatography on silica gel using Heptane:EtOAc (1:2) as mobile phase to give N-(4-(7-chloro-1H-benzo[d]imidazol-2-yl)phenyl)-1-methyl-1H-pyrazole-5-carboxamide (145 mg, 98%).

UPLC-MS (Basic Method, 2 min): rt 1.02 min, m/z 352.0 [M+H]⁺

¹H NMR (DMSO-d₆) δ: 10.43 (s, 1H), 8.15-8.25 (m, 21H), 7.94 (d, J=8.7 Hz, 21H), 7.56 (d, J=2.0 Hz, 1H), 7.45-7.53 (m, 1H), 7.17-7.29 (m, 2H), 7.12 (d, J=2.1 Hz, 1H), 4.12 (s, 3H), 2.69 (s, 1H)

### Example 20 N-(4-(1H-benzo[d]imidazol-2-yl)phenyl)-1-methyl-1H-pyrazole-5-carboxamide

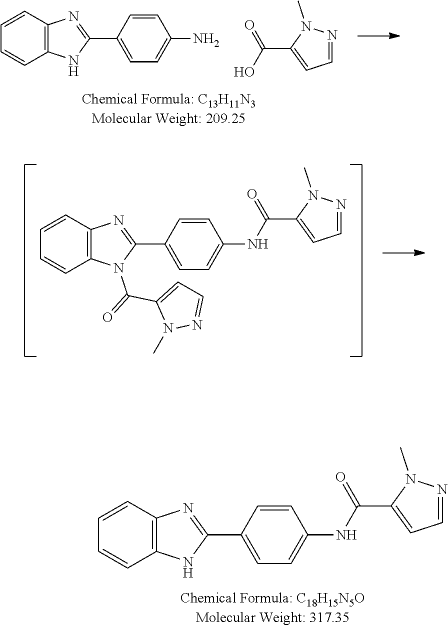

Oxalyl chloride (191 μL) was added to a solution of 1-methyl-1H-pyrazole-5-carboxylic acid (200 μL) in DCM (2 mL) and DMF (1 drop). After 1 hour, the reaction mixture was evaporated. A solution of 4-(1H-benzo[d]imidazol-2-yl)aniline (166 mg) and triethylamine (420 μL), in DCM (4 mL) was added to the initial reaction residue and stirred at ambient temp for 1 hour. The Reaction mixture was diluted with DCM and extracted sequentially with sat bicarb, water, brine, then dried over sodium sulfate, filtered and evaporated. The bis acylated material was purified on silica, eluting with a gradient of ethyl acetate (50-100%) in hexane. The pure bis acylated material was treated with ethylamine solution (2M in THF, 5 mL) for 18h. The reaction mixture was evaporated. Trituration with ethyl acetate/diethyl ether [1:4], afforded a solid which was collected by filtration (77 mg, Y=30%).

UPLC-MS (Acidic Method, 4 min): rt 1.05 min, m/z 318.1 [M+H]⁺

¹H NMR (400 MHz, DMSO-d₆) δ ppm 12.83 (br s, 1H), 10.41 (s, 1H), 8.13-8.21 (m, 2H), 7.92 (d, J=8.8 Hz, 2H), 7.64 (br d, J=5.9 Hz, 1H), 7.56 (d, J=2.0 Hz, 1H), 7.52 (br d, J=6.4 Hz, 1H), 7.19 (br d, J=4.3 Hz, 2H), 7.11 (d, J=2.0 Hz, 1H), 4.11 (s, 3H).

### Example 21 N-(4-(benzo[d]thiazol-2-yl)phenyl)-1-methyl-1H-pyrazole-5-carboxamide

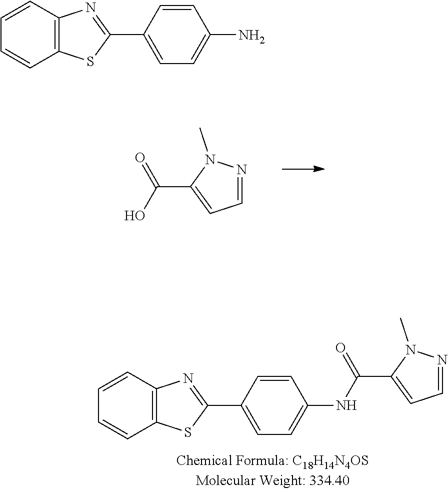

### N-(4-(benzo[d]thiazol-2-yl)phenyl)-1-methyl-1H-pyrazole-5-carboxamide

A solution of 1-methyl-1H-pyrazole-5-carboxylic acid (56 mg), HATU (168 mg), triethylamine (157 μL) were combined in THF (5 mL). After 5 min, 4-(benzo[d]thiazol-2-yl)aniline (100 mg) and a catalytic amount of DMAP were added and the resulting mixture was stirred at 65° C. for 18 h. The reaction mixture was diluted with DCM, washed with saturated solution of NaHCO₃ and brine. The organic solution was, dried over Na₂SO₄, decanted and evaporated under reduced pressure. The crude was purified by column chromatography on silica gel using 1:4 to 1:2 EtOAc:Hexane as mobile phase to give N-(4-(benzo[d]thiazol-2-yl)phenyl)-1-methyl-1H-pyrazole-5-carboxamide (25 mg, 17%).

UPLC-MS (Acidic Method, 4 min): rt 1.91 min, m/z 335.1 [M+H]⁺

¹H NMR (400 MHz, DMSO-d₆) δ ppm 10.49 (s, 1H), 8.07-8.18 (m, 3H), 8.04 (d, J=7.7 Hz, 1H), 7.92-8.00 (m, 2H), 7.51-7.60 (m, 2H), 7.42-7.50 (m, 1H), 7.13 (d, J=2.1 Hz, 1H), 4.11 (s, 3H).

### Example 22

### N-(4-(benzo[d]oxazol-2-yl)-3-methylphenyl)-1-methyl-1H-pyrazole-5-carboxamide

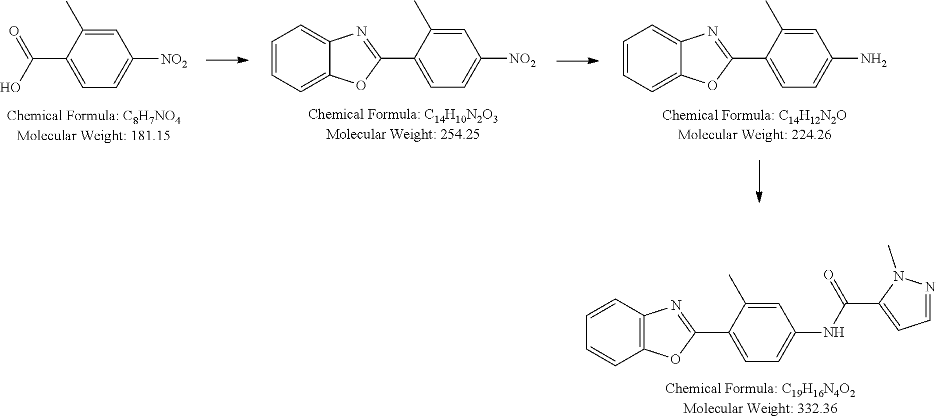 [SMILES: Cc1cc([N+](=O)[O-])ccc1C(=O)O ; Cc1cc([N+](=O)[O-])ccc1-c1nc2ccccc2o1 ; Cc1cc(N)ccc1-c1nc2ccccc2o1 ; Cc1cc(NC(=O)c2ccnn2C)ccc1-c1nc2ccccc2o1]

### 2-(2-methyl-4-nitrophenyl)benzo[d]oxazole. (General Method 4)

To a solution of 2-methyl-4-nitrobenzoic acid (1.66 g, 9.17 mmol) in DCM (15 mL) containing 5 drops of DMF was added oxalyl chloride (1.17 mL, 13.8 mmol) drop wise and the suspension was stirred at room temperature for 2 hours to form a clear solution. Volatiles were removed under vacuum and to the resulting crude acid chloride, was added a suspension of 2-aminophenol (1 g, 9.17 mmol) and triethylamine (3.3 mL, 22.9 mmol) in DCM (15 mL), drop wise, while vigorously stirring, and the reaction was continued at ambient temperature for 72 hours. The obtained suspension was filtered and the filtrate volume was reduced to dryness under vacuum, taken up in dioxane (20 mL) and refluxed for 48 hours after adding methylsulfonic acid (1.8 mL, 27.6 mmol) to it. The reaction mixture was cooled down to room temperature, diluted with DCM, washed with saturated sodium bicarbonate, brine, and the organic liquor was dried over Na₂SO₄, decanted and evaporated under reduced pressure. The crude was purified by column chromatography on silica gel using 5:1 Hexane:EtOAc as mobile phase to give 2-(2-methyl-4-nitrophenyl)benzo[d]oxazole (0.72 g, 31%). UPLC-MS (Acidic Method, 2 min): rt 1.30 min, m/z 255.1 [M+H]⁺

### 4-(benzo[d]oxazol-2-yl)-3-methylaniline

Compound prepared according to general METHOD 3 (overnight reaction) to give 4-(benzo[d]oxazol-2-yl)-3-methylaniline.

UPLC-MS (Acidic Method, 2 min): rt 1.11 min, m/z 225.1 [M+H]⁺

¹H NMR (400 MHz, DMSO-d₆) δ ppm: 7.82-7.87 (m, 1H), 7.64-7.71 (m, 2H), 7.28-7.35 (m, 2H), 6.50-6.56 (m, 2H), 5.88 (s, 2H), 2.62 (s, 3H)

### N-(4-(benzo[d]oxazol-2-yl)-3-methylphenyl)-1-methyl-1H-pyrazole-5-carboxamide (General Method 5)

A solution of 1-methyl-1H-pyrazole-5-carboxylic acid (28 mg, 0.223 mmol), HATU (84 mg, 0.223 mmol), triethylamine (95 ul, 0.67 mmol) and catalytic amount of DMAP in THF (2 mL) was stirred at room temperature for 5 minutes. 4-(benzo[d]oxazol-2-yl)-3-methylaniline (50 mg, 0.223 mmol) was added and the resulting mixture was stirred at 65° C. for 18 h. The reaction mixture was diluted with DCM, washed with saturated solution of NaHCO₃ and brine. The organic solution was, dried over Na₂SO₄, decanted and evaporated under reduced pressure. The crude was purified by column chromatography on silica gel using 1:4 to 1:2 EtOAc:Hexane as mobile phase to give N-(4-(benzo[d]oxazol-2-yl)-3-methylphenyl)isoxazole-3-carboxamide (18 mg, 24%).

UPLC-MS (Acidic Method, 4 min): rt 1.96 min, m/z 333.1 [M+H]⁺

¹H NMR (400 MHz, DMSO-d₆) δ ppm 10.45 (s, 1H), 8.16 (d, J=8.5 Hz, 1H), 7.77-7.88 (m, 4H), 7.57 (d, J=2.1 Hz, 1H), 7.37-7.46 (m, 2H), 7.13 (d, J=2.1 Hz, 1H), 4.11 (s, 3H), 2.77 (s, 3H)

### Example 23 1-methyl-N-(3-methyl-4-(4-methylbenzo[d]oxazol-2-yl)phenyl)-1H-pyrazole-5-carboxamide

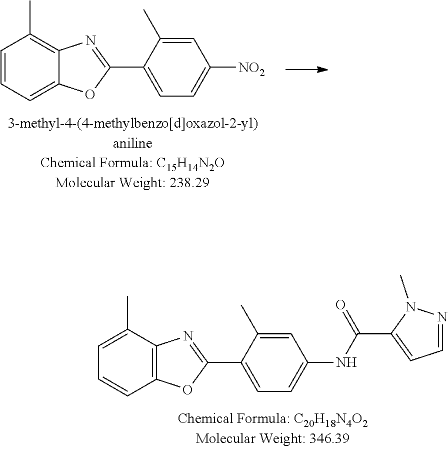 [SMILES: Cc1cc([N+](=O)[O-])ccc1-c1nc2c(C)cccc2o1 ; Cc1cc(NC(=O)c2ccnn2C)ccc1-c1nc2c(C)cccc2o1]

### 1-methyl-N-(3-methyl-4-(4-methylbenzo[d]oxazol-2-yl)phenyl)-1H-pyrazole-5-carboxamide

A solution of 1-methyl-1H-pyrazole-5-carboxylic acid (16 mg), HATU (48 mg), triethylamine (59 μL), 3-methyl-4-(4-methylbenzo[d]oxazol-2-yl)aniline (30 mg) and catalytic amount of DMAP in THF (1 mL) was stirred at 65° C. for 48 h. The reaction mixture was diluted with DCM, washed with saturated solution of NaHCO₃ and brine. The organic solution was, dried over Na₂SO₄, decanted and evaporated under reduced pressure. The crude was purified by column chromatography on silica gel using 1:4 EtOAc:Hexane as mobile phase, to give N-(3-methyl-4-(7-methylbenzo[d]oxazol-2-yl)phenyl)isoxazole-3-carboxamide (17 mg, 70%).

UPLC-MS (Acidic Method, 4 min): rt 2.19 min, m/z 347.1 [M+H]⁺

¹H NMR (400 MHz, DMSO-d₆) δ ppm 10.41 (s, 1H), 8.11-8.17 (m, 1H), 7.81-7.88 (m, 2H), 7.53-7.59 (m, 2H), 7.26-7.34 (m, 1H), 7.19-7.25 (m, 1H), 7.13 (d, J=2.0 Hz, 1H), 4.11 (s, 3H), 2.78 (s, 3H), 2.59 (s, 3H).

### Example 24 1-(2-methoxyethyl)-N-(4-(7-methylbenzo[d]oxazol-2-yl)phenyl)-1H-pyrazole-5-carboxamide

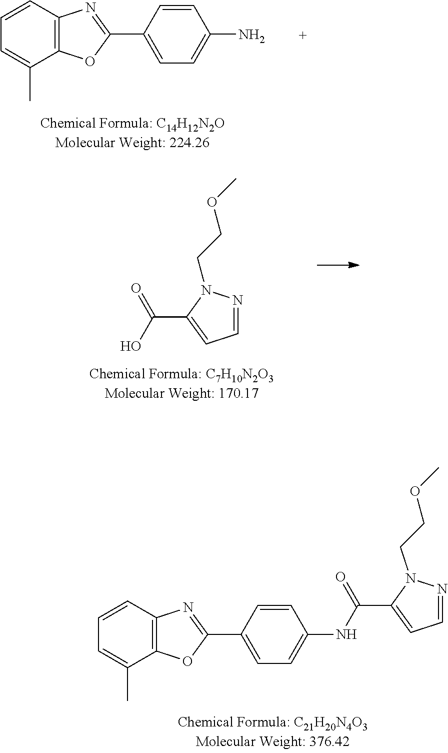 [SMILES: Cc1cccc2nc(-c3ccc(N)cc3)oc12 ; COCCn1nccc1C(=O)O ; COCCn1nccc1C(=O)Nc1ccc(-c2nc3cccc(C)c3o2)cc1]

A solution of 1-(2-methoxyethyl)-1H-pyrazole-5-carboxylic acid (101 mg, 0.59 mmol), HATU (447 mg, 1.17 mmol) and triethylamine (492 μL, 3.5 mmol) in THF (10 mL) was stirred at room temperature for 15 mins then 4-(7-methylbenzo[d]oxazol-2-yl)aniline (200 mg, 0.89 mmol) and catalytic amount of DMAP were added and the resulting mixture was stirred at 70° C. for 18 h. The reaction mixture was diluted with DCM, washed with saturated solution of NaHCO₃ and brine. The organic solution was, dried over Na₂SO₄, decanted and evaporated under reduced pressure. The crude was purified by column chromatography on silica gel using 1:3 EtOAc:Hexane as mobile phase and the obtained product was triturated in hexane:Et₂O, filtered and dried to give 1-(2-methoxyethyl)-N-(4-(7-methylbenzo[d]oxazol-2-yl)phenyl)-1H-pyrazole-5-carboxamide (107 mg, 48%). UPLC-MS (Basic Method, 4 min): rt 1.95 min, m/z 377.2 [M+H]⁺

¹H NMR (400 MHz, DMSO-d₆) δ ppm 10.57 (s, 1H), 8.17-8.28 (m, 2H), 7.91-8.05 (m, 2H), 7.56-7.64 (m, 2H), 7.27-7.32 (m, 1H), 7.21-7.26 (m, 1H), 7.08 (d, J=2.0 Hz, 1H), 4.70 (t, J=5.6 Hz, 2H), 3.69 (t, J=5.6 Hz, 2H), 3.20 (s, 3H), 2.57 (s, 3H).

The compounds of the examples were tested in selected biological assays two or more times. Data are reported as the arithmetic mean of the pIC₅₀ (−log₁₀IC₅₀) values, where IC₅₀ is defined as the concentration of compound producing a 50% inhibition of the agonist (KYNA) response.

The in vitro activity of the compounds of the present invention was assessed in the following assays:

In Vitro Assay 1: AhR Antagonism in U937 Cells (Promega P450-Glo™ Assay)

AhR antagonism was assessed in U937 cells (myeloid lineage cell line derived from a human histiocytic lymphoma). Ligand binds the AhR in the cytoplasm, and the AhR-ligand complex translocates to the nucleus and forms a heterodimer with AhR nuclear translocator (Arnt). This complex binds the xenobiotic response element (XRE) in the 5′ upstream region of the CYP1A1 promoter, enhancing CYP1A1 expression. CYP1A1 activity is subsequently determined by assessing the conversion of Luciferin-CEE to luciferin, which in turn reacts with luciferase to produce light. The amount of light produced is directly proportional to cytochrome P450 activity. U937 cells in Ultraculture serum free media (Lonza) were plated at 100,000 cells per well in a round bottom 96 well tissue culture plate. Seven concentrations of test compound (final [DMSO]1%) were added and incubated for 10 minutes before the addition of 300 μM KYNA. The plates were then placed in an incubator at 37° C., >85% humidity, 5% CO₂ for 24 hrs. After aspiration of the supernatant the CYP1A1 substrate Luciferin-CEE ([Final] 83 μM) was added and incubated for 3 hrs before the reaction was stopped by adding luciferin detection reagent and luminescence was read after 20 minutes.

In vitro assay 2: CYP1A1 inhibition assay The direct CYP1A1 inhibitory activity of test compounds was also assessed using the Promega P450-Glo™ assay system. Seven concentrations of test compound were added to a ½ area white 96 well plate. Cypex CYP1A1 bactosomes ([final] 0.5 pmol) and CYP1A1 substrate Luciferin-CEE ([final] 30 μM) were prepared in 0.1M potassium phosphate buffer and incubated with test compounds at 37° C. for 5 minutes. 0.2 mM NADPH was then added to the plates and incubated at 37° C., for 10 minutes. The reaction was stopped by adding luciferin detection reagent and luminescence was read after 20 minutes.

| | U937 | cyp1A1 |
|---|---|---|
| Example No. | pIC₅₀ | pIC₅₀ |
| 1 | 7.20 | 6.60 |
| 2 | 7.00 | 6.10 |
| 3 | — | 5.90 |
| 4 | 7.10 | 6.00 |
| 5 | 7.80 | 6.00 |
| 6 | 7.40 | 6.20 |
| 7 | 7.10 | 6.20 |
| 8 | 7.10 | 6.55 |
| 9 | 8.10 | 6.80 |
| 10 | 7.60 | 6.70 |
| 11 | 7.90 | 6.10 |
| 12 | 7.30 | 6.60 |
| 13 | 7.00 | 6.10 |
| 14 | 6.70 | 5.60 |
| 15 | 6.40 | 6.00 |
| 16 | 7.80 | 5.60 |
| 17 | 7.50 | 6.00 |
| 18 | 6.44 | 6.6 |
| 19 | 6.84 | 6.5 |
| 20 | 6.80 | 6.65 |
| 21 | 7.30 | 6.50 |
| 22 | 8.10 | 6.70 |
| 23 | 7.76 | 5.90 |
| 24 | 7.20 | 6.20 |

1-23. (canceled)

24. A compound of formula (I): 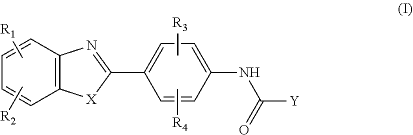
wherein
R¹ is H, C₁₋₃ alkyl, —C(O)OC₁₋₃alkyl, —C(O)NR⁵R⁶C₁₋₃ alkyl, Halogen, C₁₋₃hydroxyalkyl, —CN, C₁₋₃haloalkyl or C₁₋₃alkoxy;
R² is H, C₁₋₃ alkyl, —C(O)OC₁₋₃alkyl, —C(O)NR⁵R⁶C₁₋₃ alkyl, Halogen, (—CH₂)ₙOH, or —CN;
R³ is H, C₁₋₃ alkyl or Halogen;
R⁴ is H, C₁₋₃ alkyl or Halogen;
R⁵ is H or C₁₋₃alkyl;
R⁶ is H or C₁₋₃ alkyl;
R⁷ is H or C₁₋₃ alkyl;
R⁸ is selected from: no substituent, H, C₁₋₃ alkyl and (CH₂)ₘOC₁₋₃ alkyl, and (—CH₂)ₙOH;
n is 0, 1, 2 or 3;
m is 2 or 3;
q is 1 or 2;
i) X is O and Y is a 5 or 6 membered heteroaryl comprising at least one heteroatom —NR⁸ and at least one further heteroatom independently selected from S, O or N, said heteroaryl is substituted by one, two or three groups independently selected from halogen, hydroxyl, C₁₋₆ alkyl, C₁₋₆ alkoxy, C₁₋₆ haloalkyl, amino, C₁₋₄ mono and di-alkyl amino, C₁₋₄ mono or di-acyl amino, S(O)_{q}C₁₋₆ alkyl, C₀₋₆ alkylC(O)C₁₋₆ alkyl or C₀₋₆ alkylC(O)C₁₋₆ heteroalkyl; or
ii) X is NR⁷ and Y is 1-methyl-1H-pyrazol-5-yl or 1-(2-methoxyethyl)-1H-pyrazol-5-yl; or
iii) wherein the compound of formula I is N-(4-(benzo[d]thiazol-2-yl)phenyl)-1-methyl-1H-pyrazole-5-carboxamide or a pharmaceutically acceptable salt thereof.

25. A compound or a salt thereof according to claim 24, wherein X is O.

26. A compound or salt thereof according to claim 24, wherein Y is a five membered heteroaryl.

27. A compound or salt thereof according to claim 26, wherein Y is pyrazolyl.

28. A compound or a salt thereof according to claim 26, wherein Y is 1-methyl-1H-pyrazol-5-yl or 1-(2-methoxyethyl)-1H-pyrazol-5-yl.

29. A compound or a salt thereof according to claim 24, wherein X is as shown in formula (II):

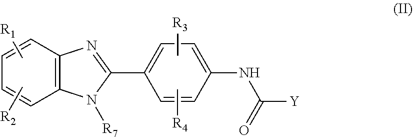

30. A compound or a salt thereof according to claim 29, wherein the R⁷ is H, —CH₃ or CH₂CH₃.

31. A compound or a salt thereof according to claim 24, wherein R² is hydrogen.

32. A compound or a salt thereof according to claim 24, wherein R⁴ is hydrogen.

33. A compound or a salt thereof according to claim 24, wherein R¹ is H, C₁₋₃ alkyl, —C(O)OC₁₋₃alkyl, —C(O)NR⁵R⁶C₁₋₃ alkyl, Halogen, —(CH₂)ₙOH, —CN, C₁₋₃haloalkyl or C₁₋₃alkoxy.

34. A compound or a salt thereof according to claim 24, wherein R¹ is selected from H, —CH₃, CO₂CH₃, CN, —C(O)NH₂, —C(O)NHCH₃, —C(O)NHCH₂CH₃, —C(O)NCH₃CH₃, Cl, —CH(CH₃)₂OH and —CH₂OH.

35. A compound or a salt thereof according to claim 24, wherein R¹ is selected from H, —CH₃, CO₂CH₃, —C(O)NH₂, —C(O)NHCH₃, —C(O)NCH₃CH₃, Cl, and —CH₂OH.

36. A compound or a salt thereof according to claim 35, wherein R¹ is selected from H, —CH₃, —C(O)₂CH₃, —C(O)NH₂, and CHOH.

37. A compound or a salt thereof according to claim 33, wherein R¹ is selected from —CH₃, —C(O)₂CH₃, —C(O)NH₂, and CHOH.

38. A compound or a salt thereof according to claim 24, wherein R² is selected from H or CH₃.

39. A compound or a salt thereof according to claim 24, wherein R⁸ is selected from H, —CH₃, CH₂CH₂OCH₃.

40. A compound according to claim 39, or a pharmaceutically acceptable salt thereof, wherein R⁸ is H.

41. A compound of formula (III): 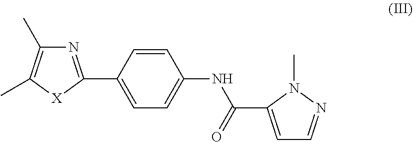
wherein X is a heteroatom selected from O, S or NR⁷, and R⁷ is H or C₁₋₃ alkyl or a pharmaceutically acceptable salt thereof.

42. A pharmaceutical composition comprising a compound of formula (I) or salt thereof according to claim 24 and an excipient, diluent or carrier.

43. A method of treating a patient comprising administering a therapeutically effective amount of a compound of formula (I) or a salt thereof according to claim 24.
